(19) United States
(12) Patent Application Publication
(10) Pub. No.: US 2022/0145316 A1
(43) Pub. Date: May 12, 2022

(54) CANCER IMMUNOTHERAPY

(71) Applicant: The Regents of the University of California, Oakland, CA (US)

(72) Inventors: Nicole Steinmetz, La Jolla, CA (US); Sourabh Shukla, La Jolla, CA (US)

(21) Appl. No.: 17/433,965

(22) PCT Filed: Feb. 28, 2020
(86) PCT No.: PCT/US20/20519

§ 371 Date: Aug. 25, 2021

Related U.S. Application Data
(60) Provisional application No. 62/812,801, filed on Mar. 1, 2019.
(60) Provisional application No. 62/926,752, filed on Oct. 28, 2019.

(51) Int. Cl. C12N 15/82; C12N 15/88; C12N 15/86; C12N 15/11

(52) U.S. Cl. CPC C12N 15/8257; C12N 15/88; C12N 15/86; C12N 2320/32; C12N 2770/40022; C12N 2770/40042; C12N 2770/40043; C12N 15/111

(57) ABSTRACT

Provided herein is a chimeric Potato virus X (PVX) filament conjugated to a therapeutic agent, a fusogenic peptide or a nucleolin-specific peptide ligand. In one aspect, they additionally comprise encapsulating a nucleic acid. Also provided are methods for use of the compositions diagnostically and therapeutically.

### CROSS-REFERENCE TO RELATED APPLICATIONS

This application claims priority under 35 U.S.C. § 119(e) to U.S. Provisional Application No. 62/812,801, filed Mar. 1, 2019, and U.S. Provisional Application No. 62/926,752, filed Oct. 28, 2019, the content of each application is hereby incorporated by reference in its entirety.

### BACKGROUND

The following discussion of the background is merely provided to aid the reader in the understanding the disclosure and is not admitted to describe or constitute prior art to the present disclosure.

Recent studies indicate that filamentous nanomaterials have tumor-homing properties that can be utilized to target and treat cancer. Decuzzi et al., Journal of Controlled Release 141, 320-327 (2010). Potato virus X (PVX) is a soft-matter filamentous nanoparticle measuring 515×13 nm. The nucleoprotein is assembled around a single stranded RNA molecule; the RNA thus is embedded into the protein capsid and therefore protected from environmental and in vivo degradation. While antibodies have been developed to target cancers such as B-cell Lymphoma, the passive immunotherapy does not elicit a systemic or memory response. Thus, a need exists in the art for an enhanced immune response. This disclosure satisfies this need and provides related advantages as well.

### SUMMARY

This disclosure provides a chimeric Potato virus X (PVX) targeted to B cell malignancies to launch anti-tumor immunity through reprogramming the tumor microenvironment. In one aspect, the chimeric PVX capsid protein filament is conjugated to a therapeutic agent through a linker element. In one aspect the chemical conjugation is through a linker, e.g., a cathepsin-cleavable linker or using Cys-maleimide chemistry.

In another aspect, the chimeric PVX is conjugated to a fusogenic peptide ligand or a nucleolin-specific peptide ligand, and encapsulates a nucleic acid. In one aspect, the fusogenic peptide ligand comprises, or alternatively consists essentially of, or yet further consists of a TAT ligand.

In one aspect, the therapeutic agent is a chemotherapeutic or anti-tumor or cancer drug. In a further aspect, the drug treats or inhibits the progression of one or more of hematological malignancies and disseminated metastases, malignant B cells, lymphoma, Non-Hodgkin's B cell lymphoma (NHL) and metastatic diseases thereof. In a further aspect, the drug is Monomethyl auristatin E ((S)-N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide or MMAE) or vcMMAE (the MMAE derivative with a valine-citrulline (Vc) linker (see medkoo.com/products/12975?gclid=EAIaIQobChMI3q3Vvvew5QIVhIbACh3QCw-rEAAYAyAAEgKRBPD_BwE, last accessed on Oct. 22, 2019). In one aspect the chemical conjugation is through a linker, e.g., a cathepsin-cleavable linker or using Cys-maleimide chemistry.

In one aspect, the nucleolin-specific peptide ligand comprises, or alternatively consists essentially of, or yet further consists of a nucleolin-specific F3 ligand. The therapeutic agent, fusogenic or nucleolin-specific peptide ligand can be chemically conjugated or genetically fused to the PVX protein filament. In one aspect the chemical conjugation is by a linker, examples of such include are known in the art, described herein and include a cathepsin-cleavable linker or using Cys-maleimide chemistry.

In yet another aspect, the nucleic acid encapsulated within the chimeric PVX comprises, or alternatively consists essentially of, or yet further consists of one or more of: a non-coding RNA, an mRNA, an siRNA, or a miRNA. In one embodiment, the mRNA encapsulated within the chimeric PVX comprises, or alternatively consists essentially of, or yet further consists of an mRNA coding for a cytokine, an immunomodulatory molecule, or a reporter protein. In one particular embodiment, the mRNA encodes the cytokine Granulocyte-macrophage colony-stimulating factor (GM-CSF). In yet a further embodiment, the mRNA encapsulated within the chimeric PVX codes for enhanced green fluorescent protein (EGFP) or mCherry reporter proteins. In yet further embodiments, the miRNA or siRNA encapsulated within the chimeric PVX comprises, or alternatively consists essentially of, or yet further consists of an miRNA or an siRNA that reprograms B-cell lymphoma cells or the tumor microenvironment and initiates cell death. In one particular aspect, the chimeric PVX of disclosed herein further comprises, or alternatively consists essentially of, or yet further consists of a detectable marker or a purification marker.

Further provided herein are isolated polynucleotides the chimeric PVX of this disclosure or an equivalent thereof, and optionally operatively linked to a promoter and/or enhancer element.

In some embodiments, the isolated nucleic acid of this disclosure sequence is comprised in a vector. For the production of vectors, the vector genome is expressed from a DNA construct encoding it in a host cell. In one aspect, the vector is a plasmid. In another aspect, the vector is a tobacco mosaic virus (TMV)-based vector.

Also provided herein are host cells comprising, or alternatively consisting essentially of, or yet further consisting of the chimeric PVX, the vector and/or the isolated polynucleotide of this disclosure. In one aspect, the host cell is a prokaryotic cell. In another aspect, the host cell is a eukaryotic cell. In one particular aspect, the host cell is a plant cell or a bacterium. In one embodiment, the host cell is an E. coli. In yet a further embodiment the host cell is a yeast or an insect cell. While in another embodiment, the host cell is an N. benthamiana cell.

Further provided herein is a method of producing the chimeric PVX of this disclosure comprising, or alternatively consisting essentially of, or yet further consisting of culturing the host cell of this disclosure. In a further aspect, the PVX is conjugated to the therapeutic agent, e.g., by a cathepsin-cleavable linker or using Cys-maleimide chemistry.

In one aspect, provided herein is a composition comprising, or alternatively consisting essentially of, or yet further consisting of a carrier and one or more of the chimeric PVX or the isolated nucleic acid of this disclosure. In another aspect, provided herein is a composition comprising, or alternatively consisting essentially of, or yet further consisting of a carrier and one or more of the chimeric PVX, the isolated nucleic acid, the vector and/or the host cell of this disclosure.

Further provided herein is a method of delivering a therapeutic agent to one or more of the spleen, lymph nodes, B cell, malignant B cells and extranodal sites. It also provides methods for treating or inhibiting the progression of hematological malignancies and disseminated metastases, malignant B cells, lymphoma, Non-Hodgkin's B cell lymphoma (NHL) and metastatic diseases thereof in a patient in need thereof. In one aspect, the treatment comprises administering an effective amount of a chimeric PVX comprising a therapeutic agent such as for example, a chemotherapeutic or anti-tumor or cancer drug. In one aspect, the drug treats or inhibits the progression of hematological malignancies and disseminated metastases, malignant B cells, lymphoma, Non-Hodgkin's B cell lymphoma (NHL) and metastatic diseases thereof. In a further aspect, the drug is MMAE or vcMMAE. In one aspect the chemical conjugation is by a cathepsin-cleavable linker or using Cys-maleimide chemistry.

The disclosure also provides a method for treating hematological malignancies and disseminated metastases, malignant B cells, lymphoma, B-cell lymphoma Non-Hodgkin's B cell lymphoma (NHL) and metastatic diseases thereof. B-cell lymphoma in a subject in need thereof by a method that comprises or alternatively consist essentially of, or yet further consists of administering to the subject the chimeric PVX and/or the composition of this disclosure. In one aspect, the subject is a human.

This disclosure also relates to methods for inhibiting the proliferation of cancer cells or cancer stem cells comprising, or alternatively consisting essentially of, or yet further consisting of contacting the cells with an effective amount of the chimeric PVX and/or the composition of this disclosure. In one embodiment, disclosed herein is a method of inhibiting the growth of a tumor and/or treating a cancer and/or preventing relapse of cancer in a subject in need thereof, comprising, or alternatively consisting essentially of, or yet further consisting of administering to the subject an effective amount of the chimeric PVX and/or the composition provided herein. In one aspect, the cancer to be treated is B-cell lymphoma.

The chimeric PVX and/or the composition provided herein may be administered either alone or in combination with diluents, known anti-cancer therapeutics, and/or with other components such as cytokines or other cell populations that are immunostimulatory. They may be administered as a first line therapy, a second line therapy, a third line therapy, or further therapy. The disclosed chimeric PVX and/or composition may be combined with other therapies (e.g., chemotherapy, radiation, surgery etc.). Non-limiting examples of additional therapies include chemotherapeutics or biologics. Appropriate treatment regimens will be determined by the treating physician or veterinarian.

In a further aspect, provided herein is a method for stimulating an immune response to a cancer or tumor cell population, the method comprising, or alternatively consisting essentially of, or yet further consisting of administering to the subject the chimeric PVX and/or composition of this disclosure in an amount effective to stimulate the immune response. In one aspect, the subject has, has had or is in need of treatment for cancer or tumor. In another aspect, the cancer is characterized as being hyporesponsive. In a further aspect, a method for stimulating an immune response to a cancer or tumor cell is provided, the method comprising, or alternatively consisting essentially of, or yet further consisting of contacting the target cell population with the chimeric PVX and/or composition of the disclosure, wherein the contacting is in vitro or in vivo. In one aspect, the cancer is B-cell lymphoma.

Also provided herein is a method of providing anti-tumor immunity in a subject, the method comprising, or alternatively consisting essentially of, or yet further consisting of administering to the subject the chimeric PVX and/or composition provided herein in an amount effective to provide the immunity to the subject. The chimeric PVX and/or composition are provided to prevent the symptoms or cancer from occurring in a subject that is predisposed or does not yet display symptoms of the cancer. The present disclosure also provides methods for using the chimeric PVX and/or composition disclosed herein in the diagnosis of cancer in a subject.

Further provided herein are methods for determining if a subject is likely to respond or is not likely to therapy, comprising, or alternatively consisting essentially of, or yet further consisting of contacting a sample isolated from the patient with the chimeric PVX and/or the composition of this disclosure, and detecting a chimeric PVX-cell complex in the sample, wherein the presence of the complex indicates that the subject is likely to respond to the therapy and the absence of complex indicates that the subject is not likely to respond to the therapy. The chimeric PVX and/or the composition may be detectably labeled. Also disclosed herein are methods further comprising, or alternatively consisting essentially of, or yet further consisting of administering an effective amount of the chimeric PVX and/or the composition of the disclosure to the subject that is determined likely to respond to the therapy.

This disclosure further relates to methods for monitoring therapy in a subject, comprising, or alternatively consisting essentially of, or yet further consisting of contacting a sample isolated from the subject with the chimeric PVX and/or the composition of this disclosure, and detecting a chimeric PVX-cell complex in the sample. The method can be performed prior to and/or after administration of an effective amount of chimeric PVX and/or composition of this disclosure to the subject. In one aspect, the chimeric PVX and/or composition is detectably labeled. In another aspect, the sample comprises one or more of sputum, serum, plasma, lymph, cystic fluid, urine, stool, cerebrospinal fluid, ascite fluid, blood, or a tissue.

In one particular aspect, the present disclosure provides kits for performing the methods of this disclosure as well as instructions for carrying out the methods of the present disclosure. The kit comprises, or alternatively consists essentially of, or yet further consists of one or more of: the chimeric PVX, the isolated nucleic acid or the composition of this disclosure and instructions for use. In another aspect, the kit comprises, or alternatively consists essentially of, or yet further consists of one or more of: the chimeric PVX, isolated nucleic acid, vector or composition of this disclosure and instructions for use. In a further aspect, the instruction for use provide directions to conduct any of the methods disclosed herein.

### BRIEF DESCRIPTION OF THE DRAWINGS

FIG. 1 illustrates structure of potato virus X (PVX) and negative-stained TEM image of PVX.

FIG. 2A-FIG. 2C illustrate that RNA-templated self-assembly (FIG. 2A) enables the production of full-length TMV (FIG. 2B, 300 nm in length) or lower-aspect ratio version thereof (FIG. 2C, 60 nm in length). Any length can be programmed as a function of the length of the synthetic RNA transcript. These methods can be adapted for PVX.

FIG. 3 illustrates an exemplary synthetic gene design.

FIG. 4 illustrates size exclusion chromatography of intact TMV (black), its coat protein (grey), and a 60 nm-sized reassembled VLP (dark grey). In this example TMV was studied; and the methods can be adapted for PVX.

FIG. 5 illustrates MCF-7-EGFP knockdown using nucleolin-targeted nanoparticles delivering siRNA. Cells are grown to confluence, incubated with PEI-PEG polymeric particles, and knockdown evaluated by fluorescence microscopy. Image on the left are control cells. Image on the right are cells knocked down by nucleolin-targeted PEI-PEG-particles.

FIG. 6A-FIG. 6B illustrate nucleic acid delivery by an exemplary plant virus-like particles (VLPs). FIG. 6A: HeLa/GFP cells; and FIG. 6B: HeLa/GFP cells incubated with CCMV+eGFP siRNA for 24 hours. Some cells no longer express GFP, and fluorescently tagged siRNA can be detected inside the cell. Grey: nucleus (DAPI), Light Grey: GFP, Lightest Grey: siRNA (labeled with Cy5), Dark Grey: lysosome.

FIG. 7 illustrates N. benthamiana plant infected with GFP-tagged PVX. Photograph was taken under UV light 18 days post inoculation (dpi).

FIG. 8A-FIG. 8D illustrate PVX viral nanoparticles. FIG. 8A shows PVX nanofilaments (515×13 nm) which are composed of 1270 identical copies of a 25 kDa coat protein (green) wrapped around a single-stranded RNA (red). PVX was conjugated with Cy5 dyes via the Lys residues on the CP using NETS-chemistry (FIG. 8B). Dye conjugation was verified using denaturing SDS-PAGE gel electrophoresis (FIG. 8C). Particle integrity was confirmed using size exclusion chromatography analysis of elution profiles and A260:280 ratios of PVX and PVX-Cy5 (FIG. 8D).

FIG. 9A-FIG. 9E illustrate biodistribution and lymphoma tropism of PVX. The biodistribution and lymphoma tropism was studied by inoculating males and female NSG mice with 5×10⁶Raji-luc B lymphoma cells via intravenous injections (FIG. 9A). On day 27 post-tumor challenge, 100 μg PVX-Cy5 particles were administered via intraperitoneal injection followed by ex vivo bioluminescence and fluorescence imaging. PVX trafficking to kidneys in male (FIG. 9B) and ovaries in female mice (FIG. 9C) was compared in Raji lymphoma bearing Raji⁺ and healthy Raji⁻ mice through colocalization of bioluminescence signal (Raji-luc cells) and fluorescence (PVX-Cy5). PVX trafficking to Raji B lymphoma cells in kidney and ovaries was also confirmed by confocal microscopy performed on tissue sections stained for CD45⁺ Raji cells (FIG. 9D and FIG. 9E).

FIG. 10A-FIG. 10F illustrate cellular tropism of PVX and PVX-vcMMAE cytotoxicity. PVX tropism towards lymphoma cells was evaluated through in vitro cell binding of PVX-Cy5 particles using a panel of cells with flow cytometry (FIG. 10A); statistical analysis was performed using Ordinary one-way ANOVA (Tukey's multiple comparison tests; **** p<0.0001). vcMMAE was conjugated to PVX via the Cys residues using the maleimide chemistry leading to formation of PVX-vcMMAE (FIG. 10B). TEM was used to verify the structural integrity of PVX post modification (FIG. 10C). vcMMAE conjugation was assessed by SDS-PAGE (FIG. 10D). Cell viability assays were used to determine cytotoxicity of PVX-vcMMAE and vcMMAE pro-drug in Raji and Daudi lymphoma cells, and normal B cells derived from healthy human donor. IC₅₀ values were calculated using GraphPad Prism software (FIG. 10E and FIG. 10F).

FIG. 11A-FIG. 11E illustrate therapeutic efficacy of PVX-vcMMAE which was evaluated in Raji B cell lymphoma model in NSG mice. Mice engrafted with 1×10⁶Raji-Luc B lymphoma cells were treated six times at four-day intervals with PBS, PVX, MMAE and PVX-vcMMAE (FIG. 11A) and tumor progression was monitored using bioluminescence imaging (FIG. 11B, FIG. 11C). Treatment efficacy was measured via overall survival (FIG. 11D) and histological analysis of kidney sections were used to highlight the efficacy of various treatments (FIG. 11E). The scale bars in E are 0.2 mm. Survival curves were analyzed using the Log-Rank Mentel-Cox test using GraphPad Prism8 software. 2 mice were lost during the study; these mice had no signs of lymphoma. Whether these mice experienced treatment-related side effects is not clear; no weight loss was observed

FIG. 12 illustrates biodistribution of PVX-Cy5 in Raji⁻ and Raji⁺ male and female NSG mice.

FIG. 13A-FIG. 13B illustrates confocal microscopy of kidney (FIG. 13A) and ovary sections (FIG. 13B) from healthy NSG mice (Raji⁻ mice) injected with PVX-Lys-Cy5 shows no PVX accumulation in these tissues. Tissue sections were stained for CD45⁺ Raji cells and with DAPI nuclei staining.

FIG. 14A-FIG. 14B illustrates efficacy of PVX-vcMMAE treatment compared with soluble MMAE in Raji B cell lymphoma bearing mice (n=5). Mice were engrafted with 1×10⁶ Raji-Luc B lymphoma cells and treated three times at four-day intervals with PBS, PVX, MMAE and PVX-vcMMAE, starting at day 11 post-tumor challenges (FIG. 14A). Survival curves were analyzed using the Log-Rank Mentel-Cox test using GraphPad Prism8 software (FIG. 14B).

FIG. 15 illustrates PVX nanofilaments conjugated to Cy5 dyes via the Cys residues on the coat protein (CP) using maleimide chemistry.

FIG. 16 illustrates cellular tropism of PVX towards lymphoma cells which was further validated by in vitro cell binding studies using PVX-lys-Cy5 and PVX-cys-Cy5 particles in a panel of cells.

FIG. 17 illustrates in vitro cell binding studies of conjugated PVX in various cell lines. Mean values are provided above bars.

FIG. 18 illustrates PVX nanofilaments biodistribution. PVX nanofilaments were confined to liver and spleens in healthy mice.

FIG. 19 illustrates systemically disseminated spread of B cell lymphoma following i.v. injection.

FIG. 20 illustrates co-localization of PVX to B-cell rich regions in spleens and draining lymph nodes (dLNs).

### DETAILED DESCRIPTION

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the chimeric PVX of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term siRNA intends short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides long.

The term MicroRNAs (miRNAs) intends a class of small noncoding RNAs of about 22 nucleotides in length which are involved in the regulation of gene expression at the posttranscriptional level by degrading their target mRNAs and/or inhibiting their translation.

The term “encode” as it is applied to nucleic acid sequences refers to a polynucleotide which is the to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

“Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small secreted proteins released by immune cells that have a specific effect on the interactions and communications between the immune cells. Cytokines can be pro-inflammatory or anti-inflammatory. Non-limiting example of a cytokine is Granulocyte-macrophage colony-stimulating factor (GM-CSF), which stimulates stem cells to produce granulocytes (neutrophils, eosinophils, and basophils) and monocytes.

As used herein, the phrase “immune response” or its equivalent “immunological response” refers to the development of a cell-mediated response (e.g. mediated by antigen-specific T cells or their secretion products). A cellular immune response is elicited by the presentation of polypeptide epitopes in association with Class I or Class II MEW molecules, to treat or prevent a viral infection, expand antigen-specific B-reg cells, TC1, CD4+T helper cells and/or CD8+ cytotoxic T cells and/or disease generated, autoregulatory T cell and B cell “memory” cells. The response may also involve activation of other components. In some aspect, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells—non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

The term “transduce” or “transduction” as it is applied to the production of chimeric antigen receptor cells refers to the process whereby a foreign nucleotide sequence is introduced into a cell. In some embodiments, this transduction is done via a vector.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

An “effective amount” or “efficacious amount” refers to the amount of an agent or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

As used herein, a “cancer” is a disease state characterized by the presence in a subject of cells demonstrating abnormal uncontrolled replication and may be used interchangeably with the term “tumor.”

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, or stomach cancer.

As used herein, the term “hematologic malignancy” refers to cancers with hematopoietic origin. In some instances, the hematologic malignancy is a B-cell malignancy. In some instances, the hematologic malignancy is a lymphoma, optionally a B-cell lymphoma. Exemplary hematologic malignancies include, but are not limited to, Diffuse large B-cell lymphoma (DLBCL), follicular lymphoma, chronic lymphocytic leukemia (CLL), small lymphocytic lymphoma (SLL), mantel cell lymphoma (MCL), marginal zone lymphomas, Burkitt lymphoma, Waldenström macroglobulinemia, hairy cell leukemia (HCL), primary central nervous system (CNS) lymphoma, or primary intraocular lymphoma.

The term “B-cell lymphoma or leukemia” refers to a type of cancer that forms in issues of the lymphatic system or bone marrow and has undergone a malignant transformation that makes the cells within the cancer pathological to the host organism with the ability to invade or spread to other parts of the body.

In some embodiments, the hematologic malignancy is a Non-Hodgkin's B cell lymphoma (NHL).

In some cases, the hematologic malignancy is a metastatic hematologic malignancy. In some cases, the hematologic malignancy is a disseminated metastasis.

In some cases, the hematologic malignancy is a relapsed or refractory hematologic malignancy.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

As used herein, the term “purification marker” or “reporter protein” refer to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Tex. Red, rhodamine, Alexa fluors, FITC, TRITC or any other fluorescent dye or hapten.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

The terms “fusion” or “chimeric” and grammatical variations thereof, when used in reference to a molecule, such as a PVX, means that a portions or part of the molecule contains a different entity distinct (heterologous) from the molecule as they do not typically exist together in nature. That is, for example, one portion of the fusion or chimera, such as PVX, includes or consists of a portion that does not exist together in nature, and is structurally distinct.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

“Cryoprotectants” are known in the art and include without limitation, e.g., sucrose, trehalose, and glycerol. A cryoprotectant exhibiting low toxicity in biological systems is generally used.

A non-coding RNA (ncRNA) is an RNA molecule that is not translated into a protein. Non-limiting examples of non-coding RNA include transfer RNAs (tRNAs) and ribosomal RNAs (rRNAs), microRNAs, siRNAs etc.

“Fusogenic peptides” are known in the art. They enable cytoplasmatic cargo delivery of non-viral vectors by aiding in endolysosomal escape. Non-limiting examples of fusogenic peptides are provided in Oliveira et al. (2007) M. Int J Pharm, 331, (2), 211-4, Hatakeyama et al. (2009) J Control Release 139, (2), 127-32, Nishimura et al. (2014) Nanobiotechnology 12, 11 and Wadia et al. (2004) S. F. Nat Med 10, (3), 310-5.

“Nucleolin-specific peptides” are known in the art. Nucleolin-specific peptides target nucleolin, a chaperone protein capable of ferrying nanoscale cargo from the cell surface into the cell.²⁶⁻³³ This only occurs in cells expressing surface nucleolin, which includes cancer cells.

### MODES OF CARRYING OUT THE DISCLOSURE

Plant viral nanofilaments, including the potato virus X, may be used as platforms for diagnostics and therapeutics. PVX and other plant viral nanoparticles or nanofilaments can be engineered with targeting ligands, drugs and/or imaging molecules. Pokorski, J. K. & Steinmetz, N. F., Mol Pharm 8, 29-43 (2011). PVX may be engineered and tailored for desired applications through genetic modification or bioconjugate chemistry. PVX nanofilaments measure 515×13 nm (AR 40), and consist of 1270 identical coat protein units. Essentially all 1270 coat proteins can be chemically addressed via their reactive Lys side chains using NHS chemistry. Steinmetz et al, Nano Lett, 10, 305-12 (2010). This disclosure provides a chimeric PVX for use in diagnosing and treating cancers such as B-cell lymphoma.

### Chimeric PVX and Isolated Polynucleotides

This disclosure provides a chimeric Potato virus X (PVX) targeted to a hematologic malignancy, e.g., the spleen, lymph nodes, and/or B cells of a hematologic malignancy (e.g., B cell malignancies, NHL, extranodal sites, and/or metastatic dissemination thereof) to launch anti-tumor immunity through reprogramming the tumor microenvironment (TME). In some embodiments, the chimeric PVX comprises a heterologous RNA, e.g., a non-coding RNA, an exogenous mRNA, an siRNA, or an miRNA. In some instances, the chimeric PVX minimizes the presence of foreign nucleic acids, thereby complies with FDA regulations. In some cases, the chimeric PVX minimizes toxicity associated with presence of foreign nucleic acids. In additional cases, the chimeric PVX capsid protein filament is conjugated to a therapeutic drug or a fusogenic or a nucleolin-specific peptide ligand. In further cases, the chimeric PVX comprising the additional therapeutic drug provides enhanced efficacy compared to endogenous PVX conjugated to the same therapeutic drug and compared to the therapeutic drug as a single agent.

In some embodiments, the chimeric PVX comprises an endosomolytic moiety, e.g., a fusogenic peptide ligand, or a nucleolin-specific peptide ligand. In one aspect, the fusogenic peptide ligand comprises, or alternatively consists essentially of, or yet further consists of a TAT ligand or a cathepsin-cleavable linker. In another aspect, the nucleolin-specific peptide ligand comprises, or alternatively consists essentially of, or yet further consists of a nucleolin-specific F3 ligand. The fusogenic or nucleolin-specific peptide ligand can be chemically conjugated or genetically fused to the PVX protein filament. Non-limiting examples of chemical conjugation include conjugating a thiol-terminated peptide through a maleimide-PEG-NHS linker targeting lysine groups on PVX. Azide/alkyne modified peptides and PVX and click chemistry can also be used for chemical conjugation. Any bioconjugation method would be applicable. For genetic fusion, the peptide is added as N-terminal fusion in a PVX plasmid containing the entire PVX genome; chimeric PVX can be produced simply by inoculating plants with the plasmid or by agroinfiltration method.

In yet another aspect, the nucleic acid encapsulated within the chimeric PVX comprises, or alternatively consists essentially of, or yet further consists of one or more of: a non-coding RNA, an mRNA, an siRNA, or an miRNA. In some instances, the chimeric PVX comprises a non-coding RNA encapsulated within the virus with a plurality of PVX coat proteins. In some instances, the non-coding RNA is from about 30 nucleotides to about 500 nucleotides in length. In some instances, the non-coding RNA is from about 30 nucleotides to about 300 nucleotides, from about 50 to about 300 nucleotides, from about 50 to about 250 nucleotides, from about 50 to about 200 nucleotides, from about 50 to about 150 nucleotides, from about 50 to about 100 nucleotides, from about 100 to about 300 nucleotides, from about 100 to about 250 nucleotides, from about 100 to about 200 nucleotides, from about 100 to about 150 nucleotides, or from about 200 nucleotides to about 300 nucleotides in length. In some cases, the non-coding RNA is derived from a viral source, e.g., from PVX; or from a mammalian source, e.g., from the mouse genome, or from a human genome. In some cases, the non-coding RNA is also referred to as junk RNA.

In some embodiments, the chimeric PVX comprises one or more mRNA encapsulated within the virus with a plurality of PVX coat proteins. In some instances, the mRNA encapsulated within the chimeric PVX comprises, or alternatively consists essentially of, or yet further consists of an mRNA coding for a cytokine, an immunomodulatory molecule, or a reporter protein. Non-limiting examples of cytokines, include Granulocyte-macrophage colony-stimulating factor (GM-CSF), TNFα, IFN-γ, TGF-β, IL-4, IL-10, IL-13, etc. In one particular embodiment, the mRNA encodes the cytokine Granulocyte-macrophage colony-stimulating factor (GM-CSF). In yet a further embodiment, the mRNA encapsulated within the chimeric PVX codes for enhanced green fluorescent protein (EGFP) or mCherry reporter proteins.

In some embodiments, the chimeric PVX comprises one or more miRNA or siRNA encapsulated within the virus with a plurality of PVX coat proteins. In some instances, the miRNA or siRNA encapsulated within the chimeric PVX comprises, or alternatively consists essentially of, or yet further consists of an miRNA or an siRNA that reprograms B-cell lymphoma cells or the tumor microenvironment and initiates cell death. Exemplary siRNA targets include, but are not limited to, HPRT1, cyclin Dl, BCL2, MYC, and CD22ΔE12. Exemplary miRNAs include, but are not limited to, miRNA-17-92 cluster, miRNA-15a/16-1 cluster, miRNA-222, let-7f, miRNA-330, miRNA-17-5p, miRNA-106a, and miRNA-210. The GM-CSF open reading frame (ORF) can be obtained from the National Center for Biotechnology Information (NCBI) (gene bank entry AAA52578.1). In some cases, the siRNA initiates cell death in a tumor cell of the B cell malignancy. In some cases, the miRNA initiates cell death in the tumor cell.

In some embodiments, the siRNA is from about 8 nucleotides to about 50 nucleotides in length. In some instances, the siRNA is from about 10 nucleotides to about 50 nucleotides, from about 10 nucleotides to about 30 nucleotides, from about 10 nucleotides to about 25 nucleotides, from about 10 nucleotides to about 20 nucleotides, from about 10 nucleotides to about 18 nucleotides, about 12 nucleotides to about 50 nucleotides, from about 12 nucleotides to about 30 nucleotides, from about 12 nucleotides to about 25 nucleotides, from about 12 nucleotides to about 20 nucleotides, about 15 nucleotides to about 50 nucleotides, from about 15 nucleotides to about 30 nucleotides, from about 15 nucleotides to about 25 nucleotides, from about 15 nucleotides to about 20 nucleotides, about 18 nucleotides to about 50 nucleotides, from about 18 nucleotides to about 30 nucleotides, from about 18 nucleotides to about 25 nucleotides, from about 18 nucleotides to about 23 nucleotides in length. In some cases, the siRNA is about 8, 9, 10, 11, 12, 13, 14, 15, 16, 17, 18, 19, 20, 21, 22, 23, 24, 25, 30, 35, 40, 45, 50, or more in length.

In some embodiments, the miRNA is from about 8 nucleotides to about 50 nucleotides in length. In some instances, the miRNA is from about 10 nucleotides to about 30 nucleotides, from about 10 nucleotides to about 25 nucleotides, from about 10 nucleotides to about 20 nucleotides, from about 10 nucleotides to about 18 nucleotides, from about 12 nucleotides to about 30 nucleotides, from about 12 nucleotides to about 25 nucleotides, from about 12 nucleotides to about 20 nucleotides, from about 15 nucleotides to about 30 nucleotides, from about 15 nucleotides to about 25 nucleotides, from about 15 nucleotides to about 20 nucleotides, from about 18 nucleotides to about 30 nucleotides, from about 18 nucleotides to about 25 nucleotides, from about 18 nucleotides to about 23 nucleotides in length. In some cases, the miRNA is about 8, 9, 10, 11, 12, 13, 14, 15, 16, 17, 18, 19, 20, 21, 22, 23, 24, 25, 30, 35, 40, 45, 50, or more in length.

In some embodiments, to achieve efficient translation in the target cell, regulatory elements can be added: the 5′ Cap structure, a 7-methyl-guanosine residue joined to the 5′-end via a 5′-5′ triphosphate as well as a poly(A) tail (additional regulatory elements, such as internal ribosome entry sites⁴ could also be included if deemed necessary). The polyA tail can be included in the sequence and the 5′Cap can be appended to the gene either post in vitro transcription using capping enzymes (e.g. New England Biolabs) or it is also possible to obtain capped mRNA by transcription through addition of the dinucleotide m7G(5′)-ppp-(5′)G. Encapsulation of the mRNA is achieved through in vitro assembly or expression in Australian tobacco.

In some embodiments, the chimeric PVX of disclosed herein further comprises, or alternatively consists essentially of, or yet further consists of a detectable marker or a purification marker. As used herein, the term detectable marker refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I. As used herein, the term purification marker or reporter protein refer to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Tex. Red, rhodamine, Alexa fluors, FITC, TRITC or any other fluorescent dye or hapten.

Further provided herein are isolated polynucleotides encoding the chimeric PVX of this disclosure or an equivalent thereof, and optionally operatively linked to a promoter and/or enhancer element. Non-limiting examples of such promoters include T3, T7 and SP6 promoters. Non-limiting examples of such enhancers include IRES, WPRE and HPRE.

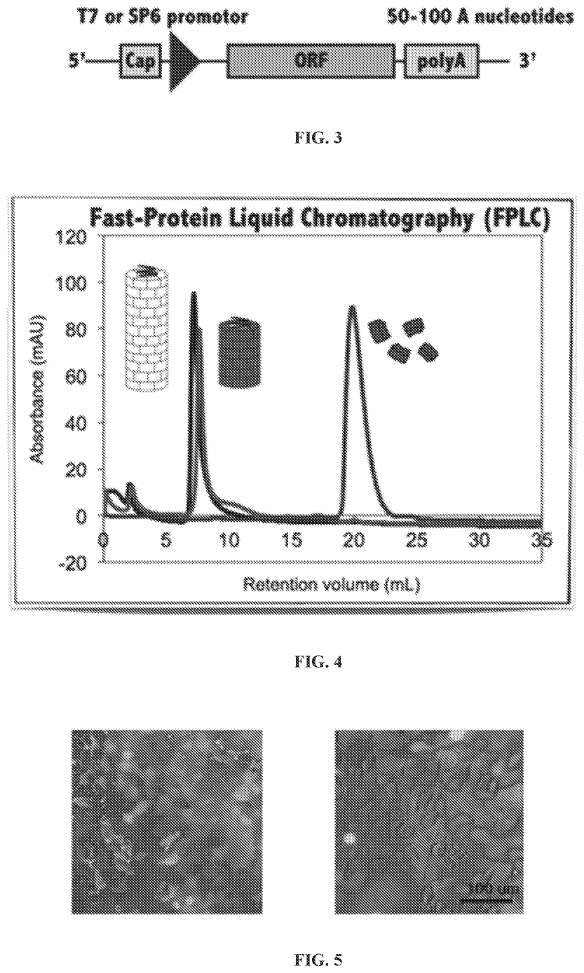

As stated above, the nucleic acid segments used in the present disclosure, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide. To achieve efficient translation in the target cell, regulatory elements can be added: the 5′ Cap structure, a 7-methyl-guanosine residue joined to the 5′-end via a 5′-5′ triphosphate² as well as a poly(A) tail³ (additional regulatory elements, such as internal ribosome entry sites⁴ could also be included if deemed necessary). The polyA tail can be included in the sequence and the 5′Cap can be appended to the gene either post in vitro transcription using capping enzymes (e.g. New England Biolabs) or it is also possible to obtain capped mRNA by transcription through addition of the dinucleotide m7G(5′)-ppp-(5′)G^{5, 6}. For in vitro transcription, a synthetic gene, e.g., as described in FIG. 3, can be cloned into a transcription plasmid, e.g. IDT Bluescript under control of a T7 or SP6 promoter (these methods are well established). The plasmid can be amplified in E. coli and transcribed using available kits, e.g. MEGAscript T7 Transcription Kit (Thermo Fisher). Based on length of the synthetic transcripts encoding EGFP and GM-CSF, PVX rods encapsulating a single copy of the gene are expected to measure only 20-30 nm in length (the length of the RNA defines the length of the nucleoprotein complex). To yield efficient assembly and to obtain higher aspect ratio particles, additional non-coding sequences can be added upstream of the OAS. As an alternative, multiple copies of the ORF could be inserted; to enable the expression of multiple ORFs from a single mRNA translational programming elements from viruses can be included such as intervening internal ribosome entry sites or leaky stop codons⁴. Multiple copies of the same mRNA and multiple different protein targets can be expressed, e.g., mRNAs encoding for EGFP and mCherry can be encapsulated within the chimeric PVX of this disclosure. The two targets are spectrally distinct and thus their expression within the same cell could be easily visualized and quantified by confocal microscopy and flow cytometry.

In some embodiments, the chimeric PVX further comprises a therapeutic agent. In some instances, the therapeutic agent is covalently bound to a solvent accessible residue, e.g., a Lys residue or a Cys residue of a coat protein of the chimeric PVX. In some instances, the therapeutic agent is covalently bound to a solvent accessible Lys residue of a coat protein of the chimeric PVX, optionally through a linker. In other instances, the therapeutic agent is covalently bound to a solvent accessible Cys residue of a coat protein of the chimeric PVX, optionally through a linker.

In some instances, each coat protein of the chimeric PVX comprises at least one therapeutic agent. In some cases, each coat protein comprises two, three, four, or more conjugated therapeutic agents.

In some embodiments, the coat protein is a full-length coat protein of the PVX. In some instances, the coat protein comprises at least 80%, 85%, 90%, 91%, 92%, 93%, 94%, 95%, 96%, 97%, 98%, 99%, or 100% sequence identity to or consists the sequence set forth in SEQ ID NO: 1, or an equivalent thereof.

(SEQ ID NO: 1)
MSAPASTTQATGSTTSTTTKTAGATPATASGLFTIPDGDFFSTARAIVAS
NAVATNEDLSKIEAIWKDMKVPTDTMAQAAWDLVRHCADVGSSAQTEMID
TGPYSNGISRARLAAAIKEVCTLRQFCMKYAPVVWNWMLTNNSPPANWQA
QGFKPEHKFAAFDFFNGVTNPAAIMPKEGLIRPPSEAEMNAAQTAAFVKI
TKARAQSNDFASLDAAVTRGRITGTTTAEAVVTLPPP
(GenBank: ALJ33138.1)

In some embodiments, the coat protein is a full-length coat protein of the PVX. In some instances, the coat protein comprises at least 80%, 85%, 90%, 91%, 92%, 93%, 94%, 95%, 96%, 97%, 98%, 99%, or 100% sequence identity to or consists the sequence set forth in SEQ ID NO: 2, or an equivalent thereof

(SEQ ID NO: 2)
MTTPANTTQAVGSTKSTTTTTAGATPANSGLFTIPDGDFFRTAKAVVASD
AVATKEELSEIQSIWKNNKVPTDTMTQAAWTLVRHCADDGSSAQTEMIGT
GPYSNGVSRARLAAAIKEVCTLRQFCKKYAPVVWNWMLTNNSPPANWQAQ
GFKPEHKFAAFDFFDGVTNPAAITPKEGLMRPPSEAEMNAAQTAAFVKIT
KARAQSNDFASLDAAVTRGRITGTTVAEAVVSLPPP
(GenBank: AAA47181.1)

In some embodiments, the coat protein is a truncated coat protein. In some instances, the truncated coat protein is a biologically functional coat protein, and is capable of assembly a ribonucleoprotein complex in the presence of an RNA, e.g., a non-coding RNA, mRNA, siRNA, or miRNA described herein. In some cases, the truncated coat protein comprises an N-terminal deletion. In some cases, the N-terminal deletion comprises a deletion of the first 2, 3, 4, 5, 6, 7, 8, 9, 10, 11, 12, 13, 14, 15, 20, 21, 22, 23, 24, 25, or more residues from the N-terminus. In some cases, the deletion is the first 2, 3, 4, 5, 6, 7, 8, 9, 10, 11, 12, 13, 14, 15, 20, 21, 22, 23, 24, 25, or more residues from the N-terminus of SEQ ID NO: 1, or an equivalent thereof. In other cases, the deletion is the first 2, 3, 4, 5, 6, 7, 8, 9, 10, 11, 12, 13, 14, 15, 20, 21, 22, 23, 24, 25, or more residues from the N-terminus of SEQ ID NO: 2, or an equivalent thereof.

In some embodiments, the therapeutic agent is an anti-cancer drug. In some embodiments, the anti-cancer drug is a small molecule drug. In some cases, the anti-cancer drug treats the B-cell malignancy described herein. In some cases, the anti-cancer drug (e.g., the small molecule drug) comprises a microtubule disrupting agent. Exemplary microtubule disrupting agents include, but are not limited to, 2-methoxyestradiol, chalcones, colchicine, combretastatin, dictyostatin, discodermolide, eleutherobin, epothilone, laulimalide, peloruside A, podophyllotoxin, taxane, cryptophycin, halichondrin B, maytansine, phomopsin A, rhizoxin, spongistatin, tubulysin, vinca alkaloid, noscapinoid, auristatin, dolastain, or derivatives or analogs thereof. In some embodiments, the anti-cancer drug is combretastatin or a derivative or analog thereof. In some embodiments, an analog of combretastatin is ombrabulin. In some embodiments, the epothilone is epothilone B, patupilone, ixabepilone, sagopilone, BMS-310705, or BMS-247550. In some embodiments, the tubulysin is a tubulysin analog or derivative such as described in U.S. Pat. Nos. 8,580,820 and 8,980,833 and in U.S. Publication Nos. 20130217638, 20130224228, and 201400363454. In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansinoid derivative or analog such as described in U.S. Pat. Nos. 5,208,020, 5,416,064, 7,276,497, and 6,716,821 or U.S. Publication Nos. 2013029900 and US20130323268. In some embodiments, the taxane is paclitaxel or docetaxel. In some embodiments, the vica alkaloid is vinblastine, vincristine, vindesine, vinorelbine, desoxyvincaminol, vincaminol, vincamajine, vineridine, vinburnine, vinpocetine, or vincamine.

In some embodiments, the anti-cancer drug (e.g., the small molecule drug) is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750, 116, 8,288,352, 8,703,714 and 8,871,720. In some embodiments, the dolastatin 15 analog is cemadotin or tasidotin.

In some embodiments, the anti-cancer drug (e.g., the small molecule drug) comprises a DNA modifying agent. In some embodiments, the DNA modifying agent comprises amsacrine, anthracycline, camptothecin, doxorubicin, duocarmycin, enediyne, etoposide, indolinobenzodiazepine, netropsin, teniposide, pyrrolobenzodiazepine, or derivatives or analogs thereof. In some embodiments, the anthracycline is doxorubicin, daunorubicin, epirubicin, idarubicin, mitomycin-C, dactinomycin, mithramycin, nemorubicin, pixantrone, sabarubicin, or valrubicin. In some embodiments, the analog of camptothecin is topotecan, irinotecan, silatecan, cositecan, exatecan, lurtotecan, gimatecan, belotecan, rubitecan, or SN-38. In some embodiments, the duocarmycin is duocarmycin A, duocarmycin B1, duocarmycin B2, duocarmycin CI, duocarmycin C2, duocarmycin D, duocarmycin SA, or CC-1065. In some embodiments, the enediyne is a calicheamicin, esperamicin, or dynemicin A.

Pyrrolobenzodiazepine (PBDs) are a class of sequence-selective DNA minor-groove binding crosslinking agents. PBD dimers are particularly potent because of their cell cycle-independent activity and because their integration minimally distorts DNA, increasing the likelihood of evasion of DNA damage repair responses.

In some embodiments, the anti-cancer drug (e.g., the small molecule drug) is pyrrolobenzodiazepine. In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120 (Table 2). In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In some embodiments, the therapeutic agent is an anti-cancer peptide or protein. As used herein, an anti-cancer peptide refers to a peptide that exerts a cytotoxic effect to a tumor cell. Exemplary peptides include, but are not limited to, peptide hormones such as luteinizing hormone-releasing hormone (LHRH) agonist and LHRH antagonist; somatostatin analogues; pituitary adenylate cyclase activating peptide (PACAP), vasoactive intestinal peptide (VIP/PACAP), and cholecystokinin (CCK). Exemplary anti-cancer proteins include, but are not limited to, cytokines and immunomodulatory proteins described herein. In some instances, the anti-cancer peptide or protein is linked to the coat protein by a peptide linker. In some cases, the anti-cancer peptide or protein and optionally the peptide linker are fused to the N-terminus of the coat protein. In some cases, the anti-cancer peptide or protein is covalently attached to the N-terminus of the coat protein, optionally by a linker.

In some embodiments, the chimeric PVX described herein comprises one or more endosomolytic moiety conjugated to a coat protein of the chimeric PVX. In some instances, the endosomolytic moiety is a cellular compartmental release component, such as a compound capable of releasing from any of the cellular compartments known in the art, such as the endosome, lysosome, endoplasmic reticulum (ER), golgi apparatus, microtubule, peroxisome, or other vesicular bodies with the cell. In some cases, the endosomolytic moiety comprises an endosomolytic polypeptide, an endosomolytic polymer, an endosomolytic lipid, or an endosomolytic small molecule.

In some cases, the endosomolytic polypeptide is a pH-dependent membrane active peptide. In some cases, the endosomolytic polypeptide is an amphipathic polypeptide. In additional cases, the endosomolytic polypeptide is a peptidomimetic. In some instances, the endosomolytic polypeptide comprises INF, melittin, meucin, or their respective derivatives thereof. In some instances, the endosomolytic polypeptide comprises INF or its derivatives thereof. In other cases, the endosomolytic polypeptide comprises melittin or its derivatives thereof. In additional cases, the endosomolytic polypeptide comprises meucin or its derivatives thereof.

In some cases, the endosomolytic moiety comprises a Bak BH3 polypeptide which induces apoptosis through antagonization of suppressor targets such as Bcl-2 and/or Bcl-xL. In some instances, the endosomolytic moiety comprises a Bak BH3 polypeptide described in Albarran, et al., “Efficient intracellular delivery of a pro-apoptotic peptide with a pH-responsive carrier,” Reactive & Functional Polymers 71: 261-265 (2011).

In some instances, the endosomolytic moiety comprises a polypeptide (e.g., a cell-penetrating polypeptide) as described in PCT Publication Nos. WO2013/166155 or WO2015/069587.

In some embodiments, the endosomolytic moiety is an endosomolytic polymer. As used herein, an endosomolytic polymer comprises a linear, a branched network, a star, a comb, or a ladder type of polymer. In some instances, an endosomolytic polymer is a homopolymer or a copolymer comprising two or more different types of monomers. In some cases, an endosomolytic polymer is a polycation polymer. In other cases, an endosomolytic polymer is a polyanion polymer.

In some instances, a polycation polymer comprises monomer units that are charge positive, charge neutral, or charge negative, with a net charge being positive. In other cases, a polycation polymer comprises a non-polymeric molecule that contains two or more positive charges. Exemplary cationic polymers include, but are not limited to, poly(L-lysine) (PLL), poly(L-arginine) (PLA), polyethyleneimine (PEI), poly[α-(4-aminobutyl)-L-glycolic acid] (PAGA), 2-(dimethylamino)ethyl methacrylate (DMAEMA), or N,N-Diethylaminoethyl Methacrylate (DEAEMA).

In some cases, a polyanion polymer comprises monomer units that are charge positive, charge neutral, or charge negative, with a net charge being negative. In other cases, a polyanion polymer comprises a non-polymeric molecule that contains two or more negative charges. Exemplary anionic polymers include p(alkylacrylates) (e.g., poly(propyl acrylic acid) (PPAA)) or poly(N-isopropylacrylamide) (NIPAM). Additional examples include PP75, a L-phenylalanine-poly(L-lysine isophthalamide) polymer described in Khormaee, et al., “Edosomolytic anionic polymer for the cytoplasmic delivery of siRNAs in localized in vivo applications,” Advanced Functional Materials 23: 565-574 (2013).

In some embodiments, an endosomolytic polymer described herein is a pH-responsive endosomolytic polymer. A pH-responsive polymer comprises a polymer that increases in size (swell) or collapses depending on the pH of the environment. Polyacrylic acid and chitosan are examples of pH-responsive polymers.

In some instances, an endosomolytic moiety described herein is a membrane-disruptive polymer. In some cases, the membrane-disruptive polymer comprises a cationic polymer, a neutral or hydrophobic polymer, or an anionic polymer. In some instances, the membrane-disruptive polymer is a hydrophilic polymer.

In some instances, an endosomolytic moiety described herein is a pH-responsive membrane-disruptive polymer. Exemplary pH-responsive membrane-disruptive polymers include p(alkylacrylic acids), poly(N-isopropylacrylamide) (NIPAM) copolymers, succinylated p(glycidols), and p(β-malic acid) polymers.

In some instances, p(alkylacrylic acids) include poly(propylacrylic acid) (polyPAA), poly(methacrylic acid) (PMAA), poly(ethylacrylic acid) (PEAA), and poly(propyl acrylic acid) (PPAA). In some instances, a p(alkylacrylic acid) include a p(alkylacrylic acid) described in Jones, et al., Biochemistry Journal 372: 65-75 (2003).

In some embodiments, a pH-responsive membrane-disruptive polymer comprises p(butyl acrylate-co-methacrylic acid). (see Bulmus, et al., Journal of Controlled Release 93: 105-120 (2003); and Yessine, et al., Biochimica et Biophysica Acta 1613: 28-38 (2003))

In some embodiments, a pH-responsive membrane-disruptive polymer comprises p(styrene-alt-maleic anhydride). (see Henry, et al., Biomacromolecules 7: 2407-2414 (2006))

In some embodiments, a pH-responsive membrane-disruptive polymer comprises pyridyldisulfide acrylate (PDSA) polymers such as poly(MAA-co-PDSA), poly(EAA-co-PDSA), poly(PAA-co-PDSA), poly(MAA-co-BA-co-PDSA), poly(EAA-co-BA-co-PDSA), or poly(PAA-co-BA-co-PDSA) polymers. (see El-Sayed, et al., “Rational design of composition and activity correlations for pH-responsive and glutathione-reactive polymer therapeutics,” Journal of Controlled Release 104: 417-427 (2005); or Flanary et al., “Antigen delivery with poly(propylacrylic acid) conjugation enhanced MHC-1 presentation and T-cell activation,” Bioconjugate Chem. 20: 241-248 (2009))

In some embodiments, the endosomolytic moiety is a lipid (e.g., a fusogenic lipid). Exemplary fusogenic lipids include 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (XTC).

In some embodiments, the endosomolytic moiety is a small molecule. Exemplary small molecules suitable as endosomolytic moieties include, but are not limited to, quinine, chloroquine, hydroxychloroquines, amodiaquins (carnoquines), amopyroquines, primaquines, mefloquines, nivaquines, halofantrines, quinone imines, or a combination thereof. In some instances, quinoline endosomolytic moieties include, but are not limited to, 7-chloro-4-(4-diethylamino-1-methylbutyl-amino)quinoline (chloroquine); 7-chloro-4-(4-ethyl-(2-hydroxyethyl)-amino-1-methylbutyl-amino)quinoline (hydroxychloroquine); 7-fluoro-4-(4-diethylamino-1-methylbutyl-amino)quinoline; 4-(4-diethylamino-1-methylbutylamino) quinoline; 7-hydroxy-4-(4-diethyl-amino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethyl-amino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethyl-amino-1-butylamino)quinoline; 4-(1-carboxy-4-diethylamino-1-butylamino) quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethyl-amino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-fluoro-4-(4-ethyl-(2-hydroxyethyl)-amino-1-methylbutylamino)quinoline; 4-(4-ethyl-(2-hydroxy-ethyl)-amino-1-methylbutylamino-) quinoline; 7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl-1)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino) quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino-6-methoxydihydrochloride quinoline; 1-acetyl-1,2,3,4-tetrahydroquinoline; 8-[(4-aminopentyl)amino]-6-methoxyquinoline dihydrochloride; 1-butyryl-1,2,3,4-tetrahydroquinoline; 3-chloro-4-(4-hydroxy-alpha,alpha′-bis(2-methyl-1-pyrrolidinyl)-2,5-xylidinoquinoline, 4-[(4-diethyl-amino)-1-methylbutyl-amino]-6-methoxyquinoline; 3-fluoro-4-(4-hydroxy-alpha, alpha′-bis(2-methyl-1-pyrrolidinyl)-2,5-xylidinoquinoline, 4-[(4-diethylamino)-1-methylbutyl-amino]-6-methoxyquinoline; 4-(4-hydroxy-alpha,alpha′-bis(2-methyl-1-pyrrolidinyl)-2,5-xylidinoquinoline; 4-[(4-diethylamino)-1-methylbutyl-amino]-6-methoxyquinoline; 3,4-dihydro-1-(2H)-quinolinecarboxyaldehyde; 1,1′-pentamethylene diquinoleinium diiodide; 8-quinolinol sulfate and amino, aldehyde, carboxylic, hydroxyl, halogen, keto, sulfhydryl and vinyl derivatives or analogs thereof. In some instances, an endosomolytic moiety is a small molecule described in Naisbitt et al (1997, J Pharmacol Exp Therapy 280:884-893) and in U.S. Pat. No. 5,736,557.

In some embodiments, the therapeutic agent is conjugated to a coat protein of the chimeric PVX by a linker. In some embodiments, a linker described herein is a cleavable linker or a non-cleavable linker. In some instances, the linker is a cleavable linker. In some instances, the linker is an acid cleavable linker. In some instances, the linker is a non-cleavable linker. In some instances, the linker includes homobifunctional cross linkers, heterobifunctional cross linkers, and the like. In some instances, the linker is a traceless linker (or a zero-length linker).

In some instances, the linker comprises a homobifuctional linker. Exemplary homobifuctional linkers include, but are not limited to, Lomant's reagent dithiobis (succinimidylpropionate) DSP, 3′3′-dithiobis(sulfosuccinimidyl proprionate (DTS SP), disuccinimidyl suberate (DSS), bis(sulfosuccinimidyl)suberate (BS), disuccinimidyl tartrate (DST), di sulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MB s), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such as1-(p-Azidosalicylamido)-4-(iodoacetamido)butane (AsIB), N-[4-(p-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido)butylamine (AsBA), and arginine-reactive and photoreactive cross-linkers such as p-azidophenyl glyoxal (APG).

In some instances, the linker comprises a reactive functional group. In some cases, the reactive functional group comprises a nucleophilic group that is reactive to an electrophilic group present on a therapeutic agent or the coat protein of chimeric PVX. Exemplary electrophilic groups include carbonyl groups—such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide.

In some embodiments, the linker comprises a peptide moiety. In some instances, the peptide moiety comprises at least 2, 3, 4, 5, 6, 7, 8, or more amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some instances, the linker comprises a peptide moiety such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the linker comprises Val-Cit. In some cases, the linker is Val-Cit.

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker is used to increase the molar ratio of therapeutic agent to the coat protein. In some instances, the dendritic type linker comprises PAMAM dendrimers.

In some embodiments, the linker is a traceless linker or a linker in which after cleavage does not leave behind a linker moiety (e.g., an atom or a linker group) to a coat protein, a therapeutic agent, or an endosomolytic moiety. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

In some instances, the linker is an acid cleavable linker. In some instances, the acid cleavable linker comprises a hydrazone linkage, which is susceptible to hydrolytic cleavage. In some cases, the acid cleavable linker comprises a thiomaleamic acid linker. In some cases, the acid cleavable linker is a thiomaleamic acid linker as described in Castaneda, et al, “Acid-cleavable thiomaleamic acid linker for homogeneous antibody-drug conjugation,” Chem. Commun. 49: 8187-8189 (2013).

In some embodiments, the therapeutic agent is conjugated to a coat protein of the chimeric PVX by a chemical ligation process. In some instances, the conjugation is by native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910. In some embodiments, the polynucleic acid molecule is conjugated to the binding moiety either site-specifically or non-specifically via native ligation chemistry.

In some embodiments, the conjugation reaction comprises reaction of a ketone or aldehyde with a nucleophile. In some embodiments, a conjugation reaction comprises reaction of a ketone with an aminoxy group to form an oxime. In some embodiments, a conjugation reaction comprises reaction of a ketone with an aryl or heteroaryl amine group to form an imine. In some embodiments, a conjugation reaction comprises reaction of an aldehyde with an aryl or heteroaryl amine group to form an imine. In some embodiments, a conjugation reaction comprises a Pictet-Spengler reaction of an aldehyde or ketone with a tryptamine nucleophile.

In some embodiments, a conjugation reaction described herein comprises a 1,3-dipolar cycloaddition reaction. In some embodiments, the 1,3-dipolar cycloaddition reaction comprises reaction of an azide and a phosphine (“Click” reaction). In some embodiments, the conjugation reaction is catalyzed by copper. In some embodiments, a conjugation reaction described herein results in cytokine peptide comprising a linker or conjugation moiety attached via a triazole. In some embodiments, a conjugation reaction described herein comprises reaction of an azide with a strained olefin. In some embodiments, a conjugation reaction described herein comprises reaction of an azide with a strained alkyne. In some embodiments, a conjugation reaction described herein comprises reaction of an azide with a cycloalkyne, for example, OCT, DIFO, DIFBO, DIBO, BARAC, TMTH, or other strained cycloalkyne, the structures of which are shown in Gong, Y., Pan, L. Tett. Lett. 2015, 56, 2123. In some embodiments, a 1,3-dipolar cycloaddition reaction is catalyzed by light (“photoclick”). In some embodiments, a conjugation reaction described herein comprises reaction of a terminal allyl group with a tetrazole and light. In some embodiments, a conjugation reaction described herein comprises reaction of a terminal alkynyl group with a tetrazole and light. In some embodiments, a conjugation reaction described herein comprises reaction of an O-allyl amino acid with a tetrazine and light. In some embodiments, a conjugation reaction described herein comprises reaction of O-allyl tyrosine with a tetrazine and light. In some embodiments, a conjugation reaction described herein comprises a hydrazino-Pictet-Spengler reaction. In some embodiments, the conjugation reaction comprises a Pictet-Spengler ligation.

In some embodiments, the therapeutic agent is conjugated to a coat protein of the chimeric PVX by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some embodiments, the therapeutic agent is conjugated to a coat protein of the chimeric PVX by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatized conjugating moiety to form an oxime bond. (see Axup et al., “Synthesis of site-specific antibody-drug conjugates using unnatural amino acids,” PNAS 109(40): 16101-16106 (2012)).

In some embodiments, the therapeutic agent is conjugated to a coat protein of the chimeric PVX by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some instances, the enzyme-catalyzed process comprises microbial transglutaminase (mTG). In some cases, the polynucleic acid molecule is conjugated to the binding moiety utilizing a microbial transglutaminze catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013))

### Vectors, Host Cells and Methods of Producing Chimeric PVX

In some embodiments, the isolated nucleic acid of this disclosure sequence is comprised within a vector. For the production of vectors, the vector genome is expressed from a DNA construct encoding it in a host cell. The techniques involved are known to those skilled in the art. Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

Also provided herein are host cells comprising, or alternatively consisting essentially of, or yet further consisting of the chimeric PVX, the vector and/or the isolated polynucleotide of this disclosure. In one aspect, the host cell is a prokaryotic cell. In another aspect, the host cell is a eukaryotic cell. In one particular aspect, the host cell is a plant cell or a bacterium. In one embodiment, the host cell is an E. coli. While in another embodiment, the host cell is an N. benthamiana cell. In yet a further embodiment, the host cells are yeast or insect cells.

Further provided herein is a method of producing the chimeric PVX of this disclosure comprising, or alternatively consisting essentially of, or yet further consisting of culturing the host cell of this disclosure.

### Compositions and Methods of Treatment, Diagnosis and Prognosis

In one aspect, provided herein is a composition comprising, or alternatively consisting essentially of, or yet further consisting of a carrier and one or more of the chimeric PVX or the isolated nucleic acid of this disclosure. In another aspect, provided herein is a composition comprising, or alternatively consisting essentially of, or yet further consisting of a carrier and one or more of the chimeric PVX, the isolated nucleic acid, the vector and/or the host cell of this disclosure. The chimeric PVX and polynucleotides, vectors, or host cells of the present disclosure also can be bound to many different carriers. Thus, this disclosure also provides compositions containing the chimeric PVX and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding viral nanoparticle filaments, or will be able to ascertain such, using routine experimentation.

The chimeric PVX and/or the composition of the present disclosure may be used to treat tumors and cancers. The chimeric PVX and/or the composition provided herein may be administered either alone or in combination with diluents, known anti-cancer therapeutics, and/or with other components such as cytokines or other cell populations that are immunostimulatory. They may be administered as a first line therapy, a second line therapy, a third line therapy, or further therapy. The disclosed chimeric PVX and/or composition may be combined with other therapies (e.g., chemotherapy, radiation, surgery etc.). Non-limiting examples of additional therapies include chemotherapeutics or biologics. Appropriate treatment regimens will be determined by the treating physician or veterinarian. In one embodiment, disclosed herein is a method of inhibiting the growth of a tumor and/or treating a cancer and/or preventing relapse of cancer in a subject in need thereof, comprising, or alternatively consisting essentially of, or yet further consisting of administering to the subject an effective amount of the chimeric PVX and/or the composition provided herein.

In one particular aspect, the tumor or cancer is B-cell lymphoma. In one embodiment, the tumor is a solid tumor. The solid tumor could be a melanoma, a colon carcinoma, a breast carcinoma and/or a brain tumor. In one aspect, the cancer to be treated is a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma.

Further provided herein is a method of treating B-cell lymphoma in a subject in need thereof comprise or alternatively consist essentially of, or yet further consists of administering to the subject the chimeric PVX and/or the composition of this disclosure.

The methods are useful to treat subjects such as humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). A mammal can be any age or at any stage of development (e.g., an adult, teen, child, infant, or a mammal in utero). A mammal can be male or female. In certain embodiments the subject has or is suspected of having a neoplastic disorder, neoplasia, tumor, malignancy or cancer.

The methods disclosed herein may further comprise or alternatively consist essentially of, or yet further consists of administering to the subject an anti-tumor therapy other than the chimeric PVX therapy. Accordingly, method aspects of the present disclosure relate to methods for inhibiting the growth of a tumor in a subject in need thereof and/or for treating a cancer patient in need thereof. This disclosure also relates to methods for inhibiting the proliferation of cancer cells or cancer stem cells comprising, or alternatively consisting essentially of, or yet further consisting of contacting the cells with an effective amount of the chimeric PVX and/or the composition of this disclosure. In one aspect, the cancer is B-cell lymphoma.

Further provided herein are methods for determining if a subject is likely to respond or is not likely to therapy, comprising, or alternatively consisting essentially of, or yet further consisting of contacting a sample isolated from the patient with the chimeric PVX and/or the composition of this disclosure, and detecting a chimeric PVX-cell complex in the sample, wherein the presence of the complex indicates that the subject is likely to respond to the therapy and the absence of complex indicates that the subject is not likely to respond to the therapy. The chimeric PVX and/or the composition may be detectably labeled. Also disclosed herein are methods further comprising, or alternatively consisting essentially of, or yet further consisting of administering an effective amount of the chimeric PVX and/or the composition of the disclosure to the subject that is determined likely to respond to the therapy.

This disclosure further relates to methods for monitoring therapy in a subject, comprising, or alternatively consisting essentially of, or yet further consisting of contacting a sample isolated from the subject with the chimeric PVX and/or the composition of this disclosure, and detecting a chimeric PVX-cell complex in the sample. The method could be performed prior to and/or after administration of an effective amount of chimeric PVX and/or composition of this disclosure to the subject. In one aspect, the chimeric PVX and/or composition is detectably labeled. In another aspect, the sample comprises one or more of sputum, serum, plasma, lymph, cystic fluid, urine, stool, cerebrospinal fluid, ascite fluid, blood, or a tissue.

In a further aspect, provided herein is a method for stimulating an immune response to a cancer or tumor cell population, the method comprising, or alternatively consisting essentially of, or yet further consisting of administering to the subject the chimeric PVX and/or composition of this disclosure in an amount effective to stimulate the immune response. In one aspect, the subject has, has had or is in need of treatment for cancer or tumor. In another aspect, the cancer is characterized as being hyporesponsive. The therapy can be first line, second line, third line, fourth line, fifth line and can be administered as a monotherapy or in combination with other therapies. In a further aspect, a method for stimulating an immune response to a cancer or tumor cell is provided, the method comprising, or alternatively consisting essentially of, or yet further consisting of contacting the target cell population with the chimeric PVX and/or composition of the disclosure, wherein the contacting is in vitro or in vivo. In one aspect, the cancer is B-cell lymphoma. These can be administered as a mono-therapy or a component is combination therapy. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration. In another aspect, the cancer or tumor is characterized as being hyporesponsive. In one aspect, the chimeric PVX and/or composition is selected for specific binding to the cancer or tumor cell. The cells can be from any species, e.g., a mammalian or a human cell. They can be isolated from a subject (e.g., from a biopsy) or a cultured cell.

Also provided herein is a method of providing anti-tumor immunity in a subject, the method comprising, or alternatively consisting essentially of, or yet further consisting of administering to the subject the chimeric PVX and/or composition provided herein in an amount effective to provide the immunity to the subject. The chimeric PVX and/or composition are provided to prevent the symptoms or cancer from occurring in a subject that is predisposed or does not yet display symptoms of the cancer.

In some embodiments, the chimeric PVX and/or composition may be delivered or administered into a cavity formed by the resection of tumor tissue (i.e. intracavity delivery) or directly into a tumor prior to resection (i.e. intratumoral delivery). In some embodiments, the disclosed chimeric PVX and/or composition may be administered intravenously, intrathecally, intraperitoneally, intramuscularly, subcutaneously, or by other suitable means of administration.

In some embodiments, the chimeric PVX modulates the population of tumor-infiltrating lymphocytes (TILs) within a tumor microenvironment. In some instances, the chimeric PVX increases the population of TILs within the tumor microenvironment relative to a population of TILs within a tumor microenvironment in a subject not treated with the chimeric PVX. In some cases, the chimeric PVX increases the population of TILs within the tumor microenvironment by about 2-fold, 3-fold, 4-fold, 5-fold, 6-fold, 7-fold, 8-fold, 9-fold, 10-fold, 20-fold, 30-fold, 50-fold, 100-fold, or more.

In some instances, the chimeric PVX increases a population of tumor infiltrating T cells in the TME. In some instances, the population of tumor infiltrating T cells in the TME is increased compared to a population of tumor infiltrating T cells in a TME of a subject not treated with the chimeric PVX. In some cases, the chimeric PVX increases the population of tumor infiltrating T cells within the TME by about 2-fold, 3-fold, 4-fold, 5-fold, 6-fold, 7-fold, 8-fold, 9-fold, 10-fold, 20-fold, 30-fold, 50-fold, 100-fold, or more.

In some instances, the chimeric PVX decreases a population of tumor regulatory T cells. In some cases, the chimeric PVX decreases the population of tumor regulatory T cells in the TME by about 2-fold, 3-fold, 4-fold, 5-fold, 6-fold, 7-fold, 8-fold, 9-fold, 10-fold, 20-fold, 30-fold, 50-fold, 100-fold, or more.

In some embodiments, the chimeric PVX is administered to the subject with an additional therapeutic agent. In some instances, the chimeric PVX and the additional therapeutic agent are administered simultaneously. In other instances, the chimeric PVX and the additional therapeutic agent are administered sequentially. In some cases, the chimeric PVX and the additional therapeutic agent are administered as a combination. In further cases, the chimeric PVX and the additional therapeutic agent are administered as separate dosage forms.

In some embodiments, the additional therapeutic agent comprises chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the additional therapeutic agent comprises an inhibitor of the enzyme poly ADP ribose polymerase (PARP). Exemplary PARP inhibitors include, but are not limited to, olaparib (AZD-2281, Lynparza®, from Astra Zeneca), rucaparib (PF-01367338, Rubraca®, from Clovis Oncology), niraparib (MK-4827, Zejula®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprises an immune checkpoint inhibitor. Exemplary checkpoint inhibitors include:

PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736;

PD-L2 inhibitors such as GlaxoSmithKline's AMP-224 (Amplimmune), and rHIgM12B7;

PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (Opdivo®, BMS-936558, MDX1106), AstraZeneca's AMP-514 and AMP-224, and Pidilizumab (CT-011) from CureTech Ltd;

CTLA-4 inhibitors such as Bristol Meyers Squibb's anti-CTLA-4 antibody ipilimumab (also known as Yervoy®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam;

LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12;

B7-H3 inhibitors such as MGA271;

KIR inhibitors such as Lirilumab (IPH2101);

CD137 inhibitors such as urelumab (BMS-663513, Bristol-Myers Squibb), PF-05082566 (anti-4-1BB, PF-2566, Pfizer), or XmAb-5592 (Xencor);

PS inhibitors such as Bavituximab;

and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprises pembrolizumab, nivolumab, tremelimumab, or ipilimumab.

In some cases, the additional therapeutic agent comprises an antibody such as alemtuzumab, trastuzumab, ibritumomab tiuxetan, brentuximab vedotin, ado-trastuzumab emtansine, or blinatumomab.

In some cases, the additional therapeutic agent comprises a cytokine. Exemplary cytokines include, but are not limited to, IL-Iβ, IL-6, IL-7, IL-10, IL-12, IL-15, IL-21, or TNFα.

In some embodiments, the additional therapeutic agent comprises a receptor agonist. In some instances, the receptor agonist comprises a Toll-like receptor (TLR) ligand. In some cases, the TLR ligand comprises TLR1, TLR2, TLR3, TLR4, TLR5, TLR6, TLR7, TLR8, or TLR9. In some cases, the TLR ligand comprises a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some cases, the additional therapeutic agent comprises an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprises use of allogeneic T lymphocytes with, e.g., anti-tumor activity, expansion of the T lymphocytes in vitro, and subsequent infusion of the expanded allogeneic T lymphocytes into a subject in need thereof.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials.

In some embodiments, the pharmaceutical composition and formulations described herein are administered to a subject by multiple administration routes, including but not limited to, parenteral, oral, buccal, rectal, sublingual, or transdermal administration routes. In some cases, parenteral administration comprises intravenous, subcutaneous, intramuscular, intracerebral, intranasal, intra-arterial, intra-articular, intradermal, intravitreal, intraosseous infusion, intraperitoneal, or intratechal administration. In some instances, the pharmaceutical composition is formulated for local administration. In other instances, the pharmaceutical composition is formulated for systemic administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel®PH102, Avicel® PH105, Elcema® P100, Emcocel®, Vivacel®, MingTia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorbate-20 or Tween® 20, or trometamol. [0338] Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In some embodiments, the pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously, alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

For the above methods, an effective amount is administered, and administration of the cell or population serves to attenuate any symptom or prevent additional symptoms from arising. When administration is for the purposes of preventing or reducing the likelihood of cancer recurrence or metastasis, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

The methods provide one or more of: (1) preventing the symptoms or disease from occurring in a subject that is predisposed or does not yet display symptoms of the disease; (2) inhibiting the disease or arresting its development; or (3) ameliorating or causing regression or relapse of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies e.g., surgical recession, chemotherapy, radiation. In one aspect, treatment excludes prophylaxis.

The present disclosure provides methods for using the chimeric PVX and/or composition disclosed herein in the diagnosis of cancer in a subject. In general, the higher the binding affinity of chimeric PVX and/or composition, the more stringent wash conditions can be performed in an immunoassay to remove nonspecifically bound material without removing the target polypeptide or cell. Accordingly, chimeric PVX and/or composition of the present technology useful in diagnostic assays usually have binding affinities of less than or about 10⁻⁶, or alternatively about 10⁻⁷, or alternatively about 10⁻⁸, or alternatively about 10⁻⁹, or alternatively about 10⁻¹⁰, or alternatively about 10⁻¹¹, or alternatively about 10⁻¹²M. In certain aspects, the chimeric PVX and/or composition used as diagnostic reagents have a sufficient kinetic on-rate to reach equilibrium under standard conditions in at least 12 hours, at least 5 hours, at least 1 hour, or at least 30 minutes.

The chimeric PVX and/or composition of the present disclosure can be used as diagnostic reagents for any kind of biological sample. In one aspect, the chimeric PVX and/or composition disclosed herein are useful as diagnostic reagents for human biological samples. The chimeric PVX and/or composition can be used to detect cancer cells such as B-cell lymphoma cancer cells in a variety of standard assay formats. Such formats include immunoprecipitation, Western blotting, ELISA, radioimmunoassay, flow cytometry, IHC and immunometric assays. Biological samples can be obtained from any tissue (including biopsies), cell or body fluid of a subject.

### Kits

In one particular aspect, the present disclosure provides kits for performing the methods of this disclosure as well as instructions for carrying out the methods of the present disclosure. The kit comprises, or alternatively consists essentially of, or yet further consists of one or more of: the chimeric PVX, the isolated nucleic acid or the composition of this disclosure and instructions for use. In another aspect, the kit comprises, or alternatively consists essentially of, or yet further consists of one or more of: the chimeric PVX, isolated nucleic acid, vector or composition of this disclosure and instructions for use. In a further aspect, the instruction for use provide directions to conduct any of the methods disclosed herein.

The kits are useful for detecting the presence of cancer such as B-cell lymphoma in a biological sample e.g., any bodily fluid including, but not limited to, e.g., sputum, serum, plasma, lymph, cystic fluid, urine, stool, cerebrospinal fluid, acitic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### EXAMPLES

These examples are provided for illustrative purposes only and not to limit the scope of the claims provided herein.

### Example 1

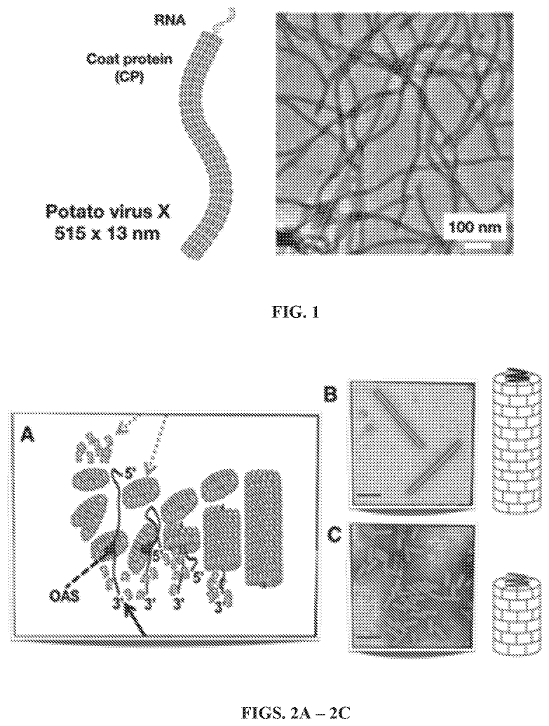

Potato virus X (PVX, FIG. 1) is a soft-matter filamentous nanoparticle measuring 515×13 nm. The nucleoprotein is assembled around a single stranded RNA molecule; the RNA thus is embedded into the protein capsid and therefore protected from environmental and in vivo degradation. Applicants data indicates that the PVX particle ‘homes’ to B cell lymphoma, and based on the immunogenic nature of the PVX particle. Without being bound by theory, Applicants hypothesize that it can be used as an immunotherapy for lymphoma treatment. At the target site, the immunostimulatory nature of the particle will lead to reprogramming of the tumor microenvironment launching anti-tumor immunity and immune memory. Key aspects are:
1. PVX homing to the disease site, lymphoma, which is achieved through interactions of the PVX protein scaffold with the cells. In ongoing studies, Applicants are deciphering the underlying mechanism of specificity. In vivo data are overwhelmingly striking showing effective targeting of metastatic disease sites.
2. The immunogenic stimulation and remodeling of the TME to launch anti-tumor immunity: this is achieved through presence of a repetitive protein capsid; the multivalency of the protein capsid is a ‘danger signal’, priming the innate immune system, therefore enabling reprogramming of the TME and launching anti-tumor immunity.
3. The immunogenic stimulation is also achieved through presence of the single-stranded RNA, which also is a ‘danger signal’ and actives innate immunity through toll-like receptor signaling.

Therefore, Applicants' disclosure provides a particle that contains RNA but that is not infectious toward plants. This can be accomplished by one or more methods:
1. PVX self-assembly around non-coding RNA which allows keeping RNA inside the PVX particle and signal through TLRs. However, in one aspect, Applicants' constructs are not coding for any proteins; so this composition would bear no risk of infection of plants or translation in mammalian cells (patients).
2. PVX self-assembly around functional RNA. In one aspect, the construct switches out the RNA with functional RNA sequences. Non-limiting examples include:
   a. mRNA coding for cytokines for additional immune stimulation
   b. mRNA coding for proteins making the tumor susceptible to other treatment regimens (e.g. overcoming drug resistance)
   c. siRNA or miRNA sequences to reprogram lymphoma cells and initiate cell death

### Design and Assembly of the Plant Viral Vector

mRNA encoding reporter proteins can be delivered using the chimeric PVX of this disclosure. Enhanced green fluorescent protein (EGFP) 1 can be used as a reporter protein for delivery. EGFP is a genetic variant of GFP with an extinction coefficient of 55,000 M-1 cm-1 and a quantum yield of 0.6 and thus suited for quantitative studies assessing the gene delivery success. mRNA encoding GM-CSF¹ can be delivered to demonstrate therapeutic protein delivery. The following genes can be designed: The EGFP or GM-CSF open reading frame (ORF) can be obtained from the National Center for Biotechnology Information (NCBI) (gene bank entry AFA52654.1 and AAA52578.1, respectively). To achieve efficient translation in the target cell, regulatory elements can be added: the 5′ Cap structure, a 7-methyl-guanosine residue joined to the 5′-end via a 5′-5′ triphosphate² as well as a poly(A) tail³ (additional regulatory elements, such as internal ribosome entry sites⁴ could also be included if deemed necessary). The polyA tail can be included in the sequence and the 5′Cap can be appended to the gene either post in vitro transcription using capping enzymes (e.g. New England Biolabs) or it is also possible to obtain capped mRNA by transcription through addition of the dinucleotide m7G(5′)-ppp-(5′)G^{5, 6}. For in vitro transcription, the synthetic gene (FIG. 3) can be cloned into a transcription plasmid, e.g. IDT Bluescript under control of a T7 or SP6 promoter (these methods are well established). The plasmid can be amplified in E. coli and transcribed using available kits, e.g. MEGAscript T7 Transcription Kit (Thermo Fisher).

PVX can be produced in N. benthamiana (an Australian tobacco species) plants. Non-limiting examples of producing chimeric PVX include 1) use of in vitro assembly methods to produce chimeric PVX particles with the PVX coat proteins encapsulating in vitro transcripts of mRNA cargos, 2) scaled-up, in planta manufacturing techniques. For in vitro assembly, PVX can be purified and disassembled into their coat protein units followed by re-assembly around the nucleic acid cargo of interest⁸. To confirm structural integrity of the produced viral vectors, transmission electron microscopy (TEM, see FIG. 1) and fast protein liquid chromatography (FPLC, FIG. 4) can be performed. Lastly, protein gel electrophoresis and western blots can be used to confirm the presence of the PVX-specific coat proteins and RT-PCR and gel electrophoresis can be used to confirm and quantify RNA encapsulation (the methods are as reported)^{9, 10}.

Based on length of the synthetic transcripts encoding EGFP and GM-CSF, PVX rods encapsulating a single copy of the gene are expected to measure only 20-30 nm in length (the length of the RNA defines the length of the nucleoprotein complex). To yield efficient assembly and to obtain higher aspect ratio particles, additional non-coding sequences can be added upstream of the OAS. As an alternative, multiple copies of the ORF could be inserted; to enable the expression of multiple ORFs from a single mRNA translational programming elements from viruses can be included such as intervening internal ribosome entry sites or leaky stop codons⁴. Multiple copies of the same mRNA and multiple different protein targets can be expressed, e.g., mRNAs encoding for EGFP and mCherry can be encapsulated within the chimeric PVX of this disclosure. The two targets are spectrally distinct and thus their expression within the same cell could be easily visualized and quantified by confocal microscopy and flow cytometry.

### Example 2—Determining the Transduction Efficiency of PVX-Delivered Nucleic Acids Vs. Contemporary Systems

To be effective for nucleic acid delivery, cytoplasmatic cargo delivery is a key requirement. Mammalian viral vectors have evolved complex machineries that enable efficient trafficking and integration¹¹⁻¹⁴. To enable cytoplasmatic cargo delivery of non-viral vectors membrane active, pH-sensitive, fusogenic peptides can be incorporated to enable endolysosomal escape¹⁵⁻¹⁸. Another strategy utilizes pH-sensitive proton polymer sponges that build up osmotic pressure within the endolysosomal compartment that eventually triggers the swelling or burst of the vesicles, resulting in cytoplasmatic cargo release¹⁹⁻²¹. Plant viruses have not evolved machineries to integrate into the mammalian cells (which provides another layer of safety). Therefore, surface chemistries can be used to induce efficient transduction. Applicants have already demonstrated the utility of fusogenic peptides conjugated to 30 nm-sized cowpea mosaic virus (CPMV) to facilitate endolysosomal escape²². Therefore, nucleic-acid loaded PVX conjugated with the fusogenic TAT motive¹⁸ can be used for efficient cargo delivery into mammalian cells; the sequence will either be genetically fused to the coat protein sequence of PVX or chemically conjugated²³. Alternative methods can target nucleolin, a chaperone protein capable of ferrying nanoscale cargo from the cell surface into the cell^{24, 25}. This only occurs in cells expressing surface nucleolin, which includes cancer cells²⁶⁻³². Using a polymeric nanoparticle Applicants have demonstrated delivery of nucleic acids and protein knockdown (FIG. 5). Cell uptake and intracellular fates of PVX-based vectors with TAT ligand or nucleolin-specific F3 ligand can also be studied³³.

To gain a better understanding of the trafficking and release of the RNA cargo, fluorescent PVX particles can be loaded with spectrally distinct RNA transcripts (e.g. using RNA labeling kits, Jena Bioscience). Cell compartments can be imaged: cell membrane (wheat germ agglutinin staining), vesicles (e.g. EEA-1 or Lamp-1 antibodies for early endosomes and endolysosomes), and nucleus (DAPI) can be stained. The TAT or nucleolin ligand density are optimized to determine any shape-dependent differences comparing PVX of various aspect ratio. This method can be used to identify a construct that delivers the mRNA cargo effectively into the cytoplasm of the cell. Imaging studies are complemented with quantitative studies: cell components can be separated (e.g. using Thermo Scientific's Cytoplasmic Extraction Kit) and quantitative qRT-PCR studies can be performed to determine payload delivery to the cytoplasm.

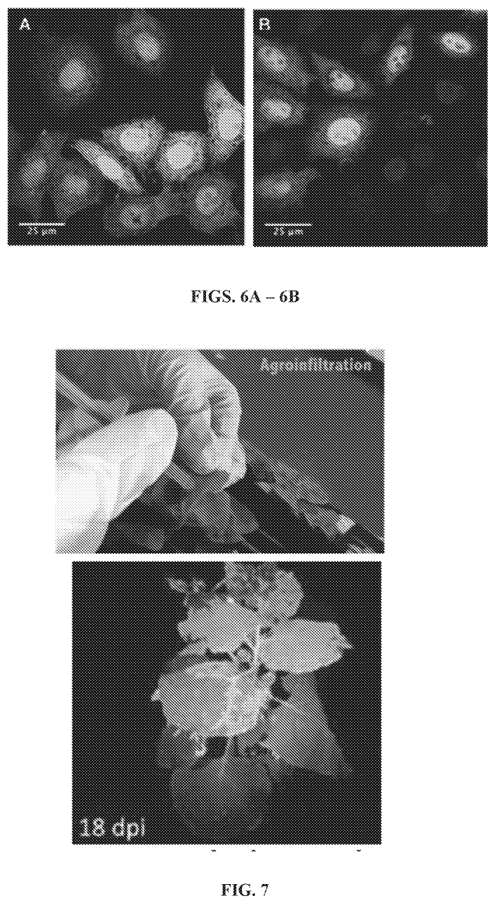

Applicants have recently demonstrated the feasibility of nucleic acid delivery using plant VLPs targeted to mammalian cells (FIG. 6). A panel of cell lines are used to determine transduction efficiency: MDA-MB-231 and MCF-7 breast cancer cells, A2780 and OVCAR3 ovarian cancer cells, and SK-MEL-3, and SH-4 melanoma cells (all cell lines are available from ATCC). To determine the transduction efficiency dose-dependency and time course studies can be established. Nucleic acid-loaded PVX vectors of distinct aspect ratio can be compared side-by-side; RNA transcripts with and without Cap and polyA tails can be delivered. The transduction efficiency can be determined using methods as described above. For EGFP and mCherry, expression levels can be quantified based on its fluorescence using flow cytometry and fluorescence imaging. GM-CSF expression can be quantified using specific antibodies and flow cytometry as well as quantitative western blots and ELISA methods^{34, 35}. When comparing viral and non-viral systems, as benchmark a lentiviral system can be used; the vector can be obtained from commercial sources such as Clonetech or ABM. As a model of non-viral gene delivery, polyplexes of DNA and polyethylenimine (PEI) can be used; PEI is a cationic polymer that combines strong DNA compaction capacity and endolysosome-lytic activity; the methods for polyplex synthesis can be adapted from literature examples³⁶⁻⁴⁰.

Lastly, a GM-CSF bioassay can be performed to determine whether the cytokine expression from transfected cells is biologically active. Supernatants from human cancer cells (see above) can be collected and GM-CSF levels can be determined using ELISA. Bioactivity can be assayed using TF-1 cells (ATCC), an erythrocyte cell line and established model system for investigation of proliferation and differentiation of myeloid progenitor cells. TF-1 cells are completely dependent on GM-CSF (and IL3). Cell proliferation can be assayed using MTT assay (e.g. Thermo Fisher) in the presence of GM-CSF or supernatant (collected from cancer cells transfected with PVX vectors) supplemented media. All cell experiments can be conducted in triplicate and repeated at least three times. Statistical data analysis can be performed using Excel software and Student's t-test. A minimum p value of <0.05 is considered significant.

### Example 3—in Plant-Production of Nucleic-Acid Loaded Plant Viral Vectors

While in vitro assembly enables the generation of various PVX-based constructs, these methods are generally not scalable. Therefore, the objective is to devise vectors for in planta production. To maximize yields a farming method that does not require chemical, post-harvest modifications of the nucleic acid delivery system can be used, therefore minimizing processing steps and maximizing scalability. Several design principles, shape and tailor-made surface chemistries can be studied to define the rules guiding effective gene delivery. The identified candidate material can 1 then be selected for scaled-up manufacture. Agrobacterium tumefaciens can be used to transfer expression cassettes to yield the desired plant viral vector. RNA loading into the PVX vector can be achieved through co-expression of the target sequence and the desired coat protein⁴¹. The coat proteins can be modified with appropriate surface coatings (TAT or F3 ligands, the latter targets nucleolin). This can be achieved either via bioconjugation of the peptide ligands post-harvest and purification of the particles or genetic incorporation as N-terminal coat protein fusion.

In fact, Applicants have already established the engineering design through fusion of GFP and other target proteins to plant viral coat proteins (FIG. 7)⁴². A binary shuttle vector can be produced through molecular cloning, and N. benthamiana plants can serve as production species^{43, 46}. Purified nucleic-acid loaded plant viral vectors with tailor-made surface chemistries can be isolated and characterized by reverse transcription (RT)-PCR followed by sequencing to confirm RNA loading, TEM to confirm particle assembly, and western blots to confirm protein expression and ratio of surface ligands. The yield of the product per kg of leaf materials can be determined using the methods described herein.

Determining shelf life and stability of the plant viral vector. To determine the shelf life of the materials produced during storage in leaf tissue or purified in aqueous buffer, samples can be stored at −80° C., −20° C., 4° C. as well as at room temperature; transfection experiments can be carried out after 1, 3, 6, and 12 months storage. One set of samples can also be selected to undergo freeze-thaw cycles to determine long-term stability of the product; in all experiments both the transfection efficiency as well as structural vector intactness can be assessed using the above-described methods.

Stability in biological media. Due to their high charge density, polyplexed non-viral systems can exhibit instability in biological media and aggregate based on interactions with serum components^{11, 47}. This is in stark contrast to the protein-based viral vectors, which are naturally stable in biological media. The zwitterionic nature of the protein coat renders the viral nanoparticle biocompatible with excellent colloidal stability and minimized interactions with plasma proteins⁴⁸. To confirm the stability and biocompatibility of the plant viral vectors in comparison to the lentiviral vector [Clonetech or ABM] and non-viral vectors prepared from polyplexes of DNA and PEI³⁶⁻⁴⁰, the nanoparticle assemblies with and without fusogenic or nucleolin-specific peptides can be tested in PBS, physiological saline (0.15 M NaCl) and cell growth medium containing 10% (v/v) serum. Agglomeration and precipitation can be measured and dynamic light scattering can be used to determine the hydrodynamic radius of the particles over time.

### Example 4—Non-Hodgkin's Lymphomas Drug Delivery Enabled by the Malignant B Cell Tropism of Plant Viral Nanoparticle Potato Virus X

Non-Hodgkin's B cell lymphomas (NHL) include a diverse set of neoplasms that constitute ˜90% of all lymphomas and constitute the largest subset of blood cancers. While chemotherapy is the first line of treatment, the efficacy of contemporary chemotherapies is hampered by dose-limiting toxicities. The suboptimal dosing leads to partial remission and ˜40% of patients exhibit relapsed or refractory disease. Therefore more efficacious drug delivery systems are needed to improve treatment outcomes in NHL patients.

Drug delivery approaches can be utilized to reduce systemic adverse effects associated with chemotherapy; and drug targeting can achieve larger chemotherapy doses to be delivered to the cancer cells. In some instances, contemporary drug delivery approaches are based on antibody-drug conjugates (ADCs) where monoclonal antibodies targeting tumor antigens deliver chemotherapy selectively to cancer cells. Despite the potential of ADCs, only four ADCs have been approved by the FDA. ADC development is hindered by a complex manufacturing process, low drug payloads and limited repertoire of candidate drugs. Additionally, physiological complexities including targeted receptor heterogeneity and down regulation in tumors, recycling of ADCs with receptors leading to non-specific drug release and premature release of drugs due to typically long circulation half lives of these antibodies (days to weeks) further hamper the success of ADCs.

In some embodiments, nanoparticle-based drug delivery systems are utilized to overcome some of these deficiencies. For example, nanoparticles enable the delivery of larger therapeutic payloads and the manufacture and bioconjugation schemes are scalable to incorporate a broad range of chemotherapy while improving drug stability and bioavailability. However, many cancer nanomedicines rely on passive tumor homing based on the enhanced permeability and retention (EPR) effect, which is controversial because of tumor heterogeneity. Furthermore, this approach is ineffective in low EPR tumors, metastatic lesions and hematological malignancies such as NHL. Therefore, to treat NHL, active targeting strategies must be devised. Contemporary approaches focused largely on targeting to overexpressed B cell antigens such as CD20/CD22. Nevertheless, such targeting strategies are also subject to the receptor heterogeneity and possible down regulation due to selection pressures as a result of treatment.

This study provides an illustrative example that nanofilaments from the plant virus potato virus X (PVX) homes to B cell lymphoma enabling drug targeting. Unlike mammalian viruses, plant viruses do not have known tissue tropisms or infectivity in mammals. Unexpectedly, the data indicate an intrinsic affinity of PVX towards B cell lymphoma in mouse models of orthotropic human lymphoma that enables efficient delivery of chemotherapy to malignant B cells leading to a reduction in tumor progression and improved survival.

Cell lines and Reagents: Cell lines (Raji, Daudi, OCI-AML3, OVCAR-3, HCT116) were maintained in RPMI media supplemented with 10% (v/v) cosmic serum (GE Healthcare) and 1% (v/v) pencillin-streptomycin (GE Healthcare). Luciferase expressing Raji cells (Raji-luc) were generated by stably transfecting Raji cells using the plasmid pLenti-CMV V5-luciferase (Addgene) using methods as reported in Campeau, et al., PLoS One, 4(8): e6529 (2009). PBMCs were isolated from healthy donor blood obtained from the Hematopoietic Biorepository and Cellular Therapy Core at CWRU and maintained in RPMI media supplemented with 10% (v/v) cosmic serum (GE Healthcare) and 100 U/mL Penicillin with 100 μg/mL Streptomycin (GE Healthcare). Monomethyl auristatin E (MMAE) and vcMMAE were obtained from MedChemExpress, (Monmouth Junction, N.J.).

PVX production: PVX was propagated in Nicotiana benthamiana plants and purified according to the protocols disclosed in Shukla, et al., Methods Mol Biol 1776: 61-84 (2018). The yields from 100 g of infected leaves were ˜10 to 20 mg of pure PVX.

Chemical bioconjugation of Cy5 dyes: PVX-Cy5 fluorescent particles were synthesized by coupling NHS-Sulfo-Cy5 (Lumiprobe) to PVX via lysine residues. PVX (at 2 mg mL-¹) was reacted with 0.5 molar excess of NHS-sulfo-Cy5/CP in 0.1 M potassium phosphate buffer (pH 7.0) supplemented with 10% (v/v) DMSO on a rotisserie overnight at room temperature. Dye labeled PVX was purified by ultracentrifugation at 112,000×g for 3 hours over a 30% (w/v) sucrose cushion. The resulting pellet was resuspended in 0.1 M potassium phosphate buffer pH 7. Post-purification, PVX concentration and number of Cy5/PVX were determined by UV-vis spectroscopy using the Beer-Lambert law and the PVX and Sulfo-Cy5-specific extinction coefficients of 2.97 mL mg⁻¹ cm⁻¹ at 260 nm and 271,000 M⁻¹ cm⁻¹ at 647 nm, respectively. Particle integrity was verified using size exclusion chromatography using a Superose6 column on the ÄKTA Explorer chromatography system (GE Healthcare).

Chemical bioconjugation of vcMMAE: vcMMAE (MedChem Express) was conjugated to PVX via the sulfhydryl side chains on the Cysteine residues using the maleimide chemistry. 7500 molar excess of vcMMAE was reacted overnight with PVX at a protein concentration of 1 mg mL⁻¹ in presence of 10% (v/v) DMSO. PVX-vcMMAE was purified via ultracentrifugation at 112,000×g for 3 hours over 30% sucrose cushion. The recovered pellet was resuspended in 0.1 M potassium phosphate buffer pH 7. Purified PVX-vcMMAE samples were characterized by transmission electron microscopy (FEI Tecnai Spirit G2 BioTWIN Transmission Electron Microscope) for particle stability. Drug loading was quantified by SDS-PAGE (4-12% NuPAGE gels, 1×MOPS running buffer, Invitrogen) followed by densitometry analysis performed using the band analysis tool in ImageJ software.

PVX biodistribution and intra-tissue localization: All animal experiments were carried out in accordance with the Case Western Reserve University's Institutional Animal Care and Use Committee (IACUC). Male and female NOD/SCID/IL2-Rγ (NSG) mice (7 weeks; n=3) were injected with 5×10⁶ Raji-luc cells intravenously. Tumor progression was monitored using in vivo bioluminescence imagine on IVIS Spectrum imager (Perkin Elmer). On day 27 from tumor challenge, mice were injected intraperitoneally (i.p.) with 100 μg of PVX-lys-Cy5 in 200 μL of PBS and then imaged using IVIS spectrum after 24 hours for bioluminescence from Raji-luc cells and fluorescence from PVX-Cy5 particles. Post-imaging mice sacrificed, and harvested tissues were imaged for bioluminescence and fluorescence to determine lymphoma invasion and dissemination and PVX trafficking, respectively. Tissues were then frozen in OCT medium (Tissue-Tek, Sakura Finetek, USA) and stored at −80° C. for immunofluorescence and histology analysis. Tissue sections (10 μm thick) prepared on a Leica cryostat were fixed in ice-cold 95% (v/v) ethanol for 20 minutes, permeabilized using 0.2% (v/v) Triton-X in PBS for 2 min and blocked with 10% (v/v) goat serum for 1 hour. Sections were then stained with FITC-anti-CD45 antibody (Biolegend, San Diego, Calif.). Slides were mounted using Fluorshield with DAPI mounting media (Sigma) resulting in nuclear staining and sealed using nail polish and were stored at −20° C. Stained sections were imaged on Olympus Fluoview FV1000 confocal microscope.

PVX cell binding: 5 μg of PVX-Cy5 was added to 200,000 cells/well/100 μL and incubated for 1 hour on ice, in dark. Cells were washed twice with PBS with 3% FBS and analyzed by flow cytometry on Attune NxT flow cytometer. Data was analyzed using the FlowJo software version 10.6.1.

Cell viability assay: Cells were plated at a concentration of 200,000 cells/mL using a clear-bottom 96-well cell culture plate and treated with 56 nM and 167 nM doses of vcMMAE and PVX-vcMMAE for 72 hours at 37° C. in a humidified atmosphere containing 5% CO₂. Cell viability and proliferation in cell lines were estimated using a resazurin based assay (Prestoblue, Thermo Scientific). Cell viability in healthy B cells was measured using 7-AAD (Cayman Chemical Company, Ann Harbor, Mich.) cell exclusion using flow cytometric analysis gated on the B cell fraction (CD19-PE, BD Biosciences, Franklin Lakes, N.J.).

Drug delivery study and histology: NSG male mice (7 weeks) were inoculated with 1×10⁶ Raji-luc cells intravenously. Mice were randomly split into 4 groups (n=5): Vehicle (PBS), 0.125 mg kg⁻¹ MMAE, 1 mg PVX (equivalent amount of PVX protein as used in the PVX-vcMMAE treatment group), and 0.4 mg/kg PVX-vcMMAE. For the first study, dosing started on day 3 and drugs/drug delivery system were injected i.p. every 4 days for a total of 6 injections. For the second study, dosing started on day 11 and drugs/drug delivery system were injected i.p. every 4 days for a total of 3 injections. For both studies, weekly bioluminescence imaging using the IVIS Spectrum was used to monitor lymphoma progression. Survival of the mice was monitored and documented daily and mice were sacrificed when they either lost >15% of their body weight or showed signs of disease burden (hind leg paralysis). Tissues were harvested for histology using H&E staining to show tumor burden or lack thereof.

Statistical analysis: All statistical analysis was performed using the GraphPad Prism v8.2 software. For cell binding studies, statistical significance was determined using Ordinary one-way ANOVA and Tukey's multiple comparisons test (**** for p<0.0001; *** for p=0.0001; ** for p<0.01 and * for p<0.05). Survival data was plotted as Kaplan-Meier plot and analyzed with Log-rank (Manel-Cox) test.

Results and Discussion:

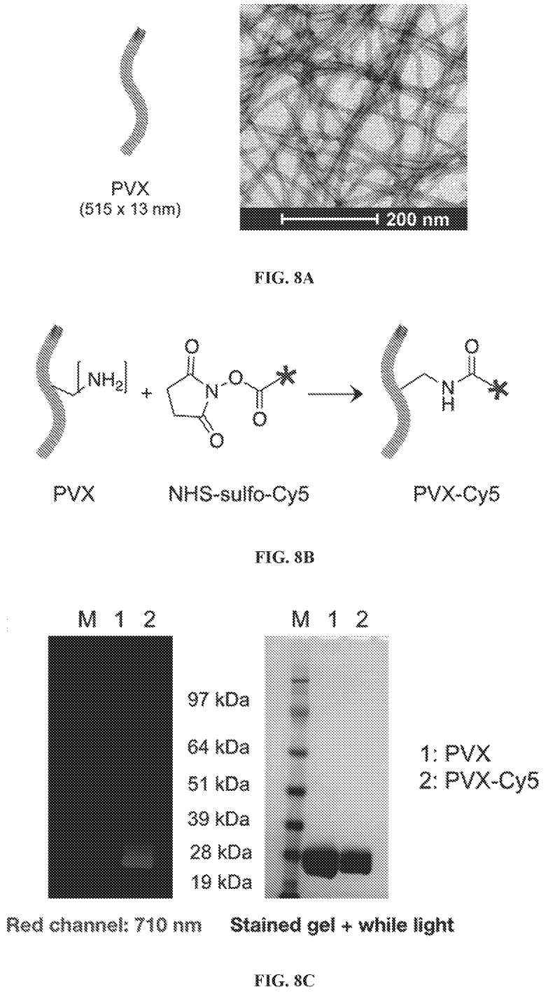

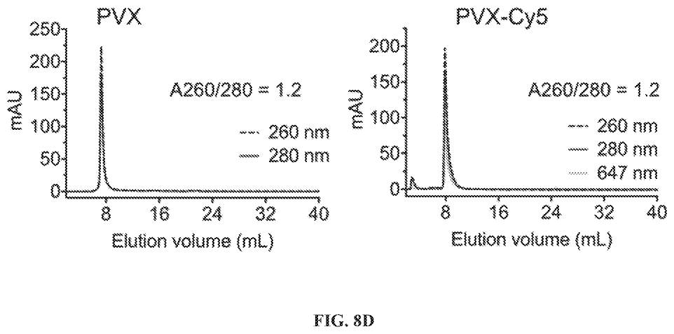

PVX was propagated in Nicotiana benthamiana plants and purified using the protocol as described in Shukla, et al., Methods Mol Biol 1776: 61-84 (2018) with yields of ˜20 mg virus from 100 g of infected leaves. PVX is a flexible filamentous nucleoprotein measuring 515×13 nm that is composed of 1270 identical copies of 25 kDa coat protein (CP) subunits (FIG. 8A). Each PVX coat protein has three solvent exposed lysines and one solvent exposed cysteine residue, which enables the biochemical conjugation of drugs, fluorescent dyes, peptides and imaging contrast agents via NHS or maleimide chemistries. It had observed that PVX (unlike other plant viruses) traffics to B cell follicles in spleen and lymph nodes in healthy mice. Therefore, it was set out to assess whether PVX further targets NHL. PVX-B lymphoma cell tropism in vivo and in vitro was evaluated using fluorescently labeled particles. PVX-Cy5 particles were synthesized by reacting NHS-sulfo-Cy5 with PVX (FIG. 8B). Using a dye/CP ratio of 0.5, ˜190 dyes were conjugated per PVX particle. Dye labeling was confirmed through SDS-PAGE gel electrophoresis with appearance of the 25 kDa fluorescent band (FIG. 8C). Post-purification stability of PVX-Cy5 particles was confirmed using size exclusion chromatography that showed a characteristic elution profile with stable 260:280 ratios for PVX and co-elution of the fluorescent dye with the 260/280 peaks (FIG. 8D).

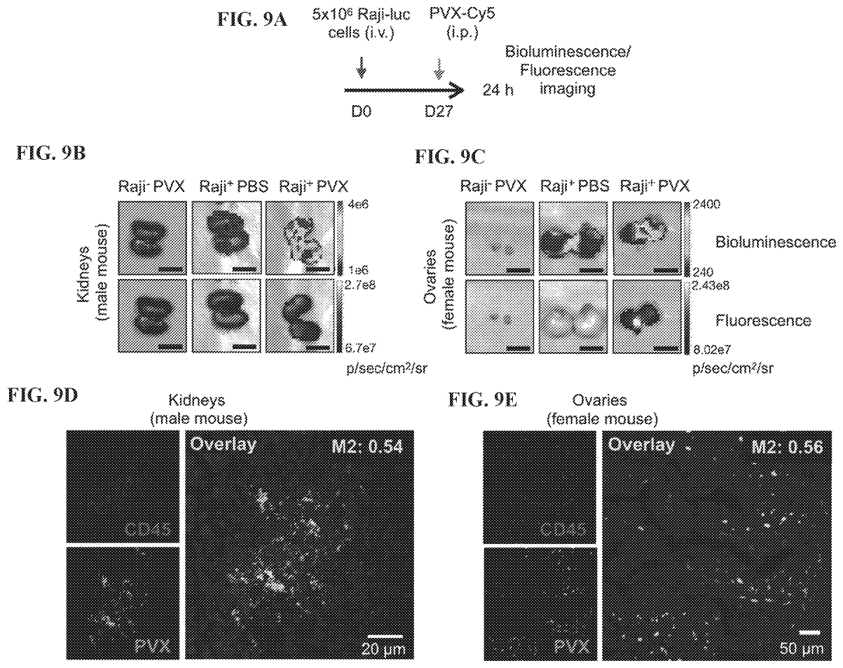

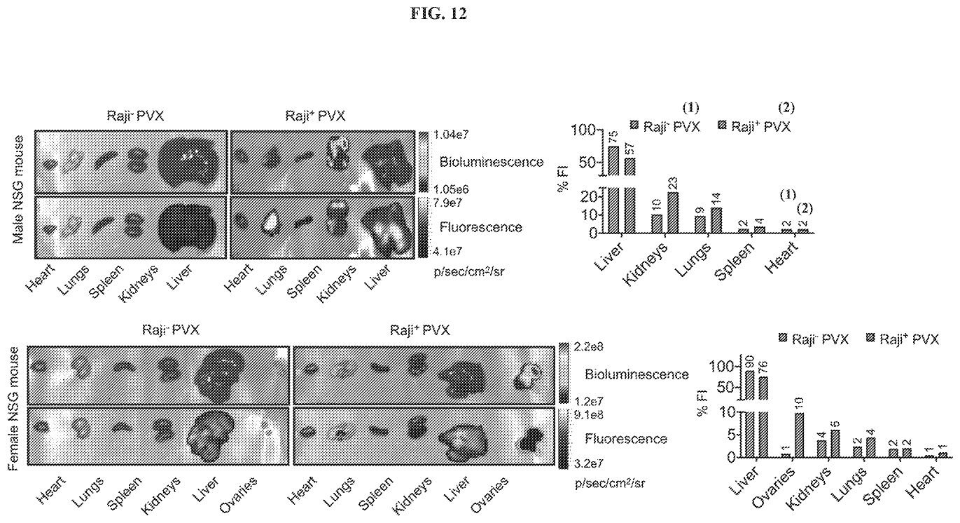

Next, PVX biodistribution was evaluated in a mouse xenograft model of human NHL. Luciferase expressing Raji cells (Raji-luc), a human Burkitt lymphoma cell line, were injected intravenously into NOD/SCID/IL2-Rγ (NSG) female or male mice to establish the disease. Tumor progression was monitored via in vivo bioluminescence imaging, and after establishment of metastatic disease was confirmed (at day 27 after Raji cell challenge), fluorescent PVX-Cy5 particles were injected intraperitoneally (i.p.) (5 mg kg⁻¹) in healthy (Raji⁻) and lymphoma bearing (Raji⁺) male and female mice (n=3) (FIG. 9A). Mice were euthanized after 24 h and major organs were harvested for ex vivo bioluminescence and fluorescence imaging. The bioluminescence imaging of harvested tissues indicated lymphoma invasion primarily in the kidneys and lungs in male mice (FIG. 9B and FIG. 12) and in the ovaries in female mice (FIG. 9C and FIG. 12). The ovaries in lymphoma bearing female mice (Raji⁺) were also enlarged as compared to healthy mice (Raji⁻) (FIG. 9C). PVX biodistribution observed through ex vivo fluorescence imaging of harvested tissues showed unexpected and interesting results: while PVX sequestration in the liver was observed as anticipated in all mice (FIG. 12), in contrast to Raji⁻ mice, the presence of PVX was observed in kidneys and ovaries of male and female Raji⁺ mice, respectively, where the fluorescent signals co-localized with the bioluminescence from Raji-Luc cells (FIG. 9B and FIG. 9C). Thus, in male Raji⁻ mice, the liver and kidneys accounted for ˜75% and ˜10% of total PVX-Cy5 fluorescence signal, in Raji⁺ mice an increased proportion of signal (˜23%) was detected from the kidneys (FIG. 12). Similarly, in female mice the percentage of PVX signal in ovaries rose sharply from 1% in Raji⁻ mice to ˜10% in Raji⁺ mice (FIG. 12).

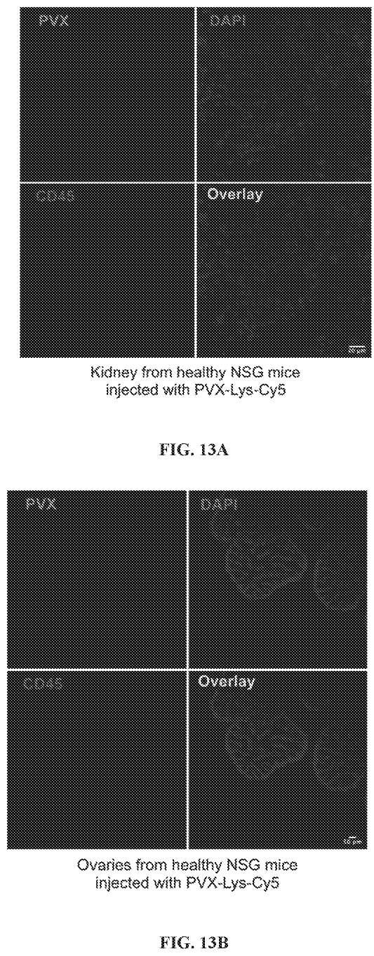

To further validate this observation, tissue sections stained with human specific anti-CD45 antibody were imaged using confocal microscopy. Both kidney and ovary sections showed some degree of co-localization of the PVX-Cy5 signals with that of human CD45⁺ Raji cells (Mander's M2 values 0.54 and 0.56, respectively) (FIG. 9D and FIG. 9E). Moreover, no PVX fluorescence was observed in the kidney and ovary sections under confocal microscopy from Raji⁻ mice (FIG. 13A-FIG. 13B), mirroring the ex vivo imaging data. Together, these results highlight the unusual trafficking of PVX to tissues harboring disseminated B lymphoma cells in the NSG mouse model of human NHL.

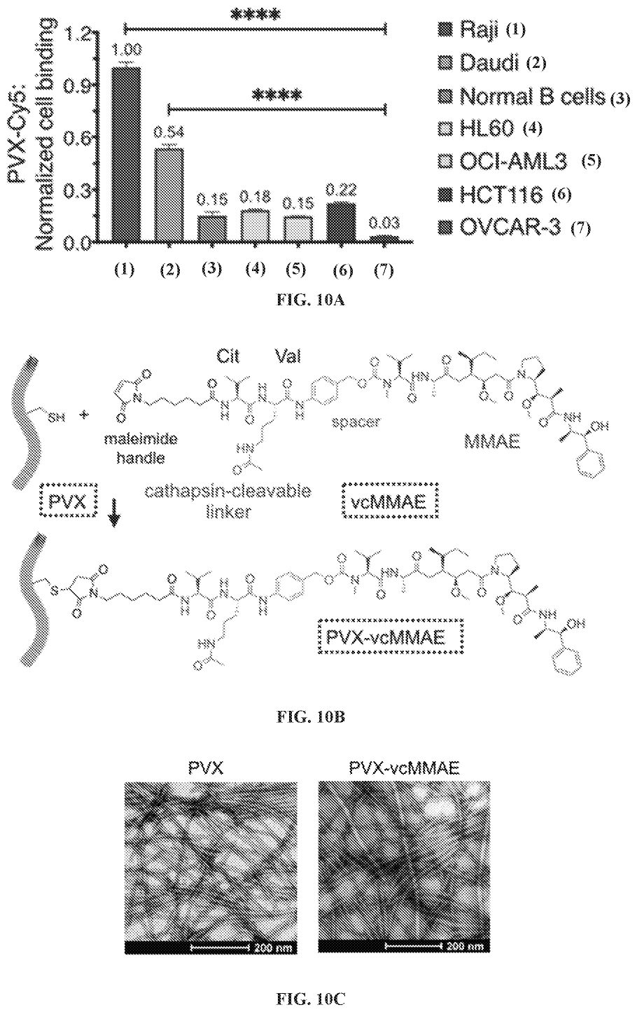

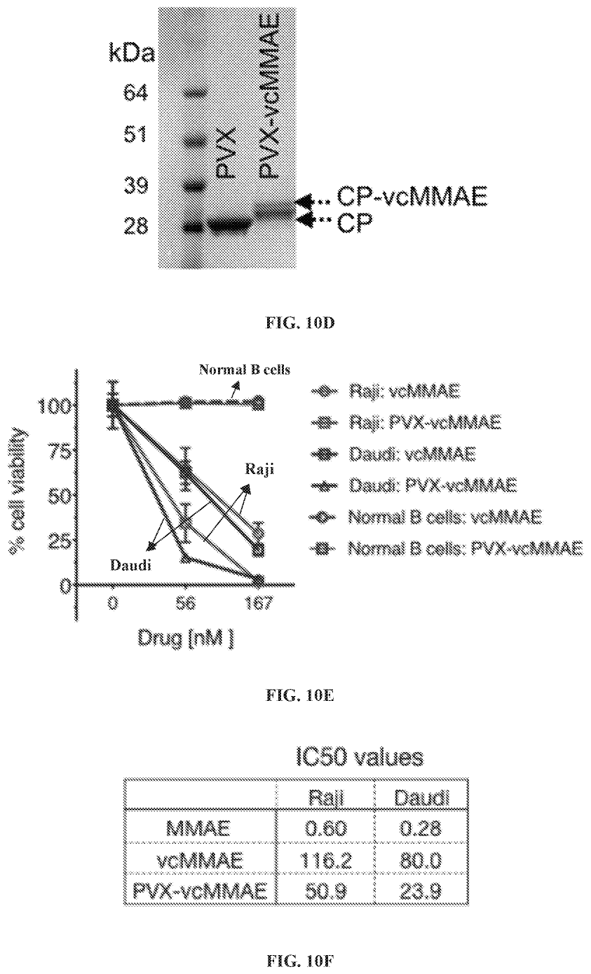

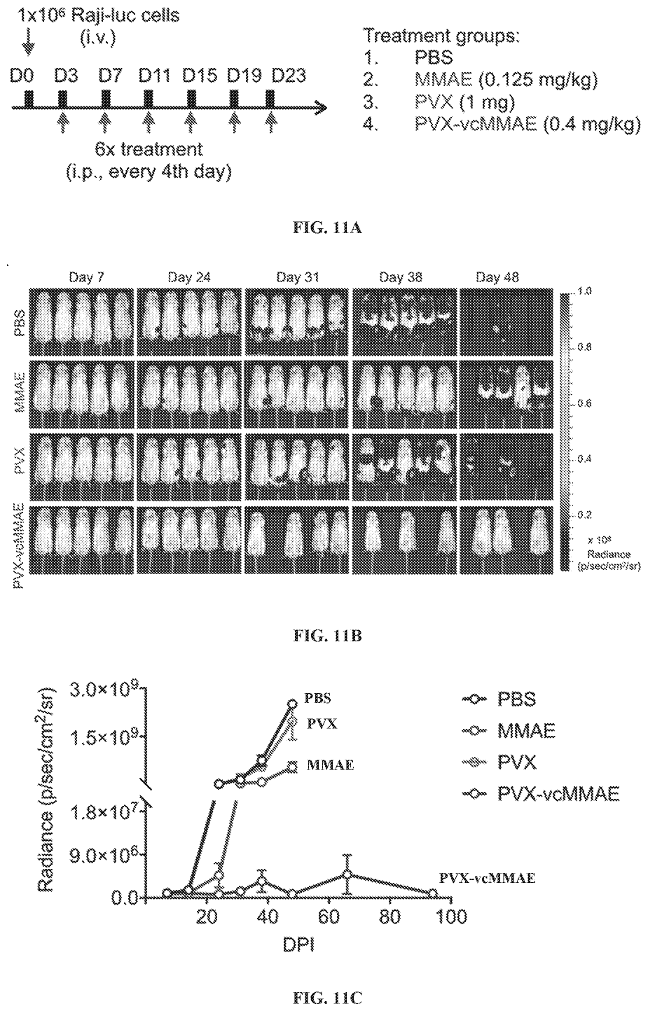

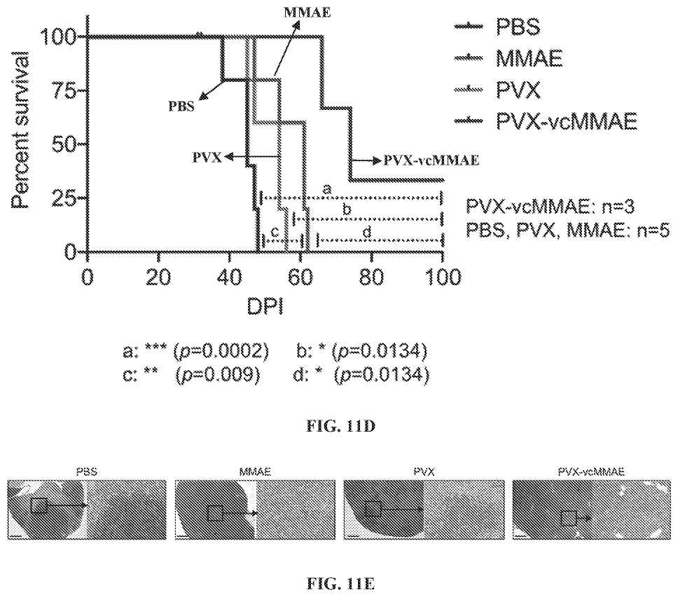

Next, the cellular tropism of PVX towards B lymphoma cells was validated in vitro. Binding of fluorescent PVX particles (PVX-Cy5) to Raji cells was compared with a panel of cell lines including Daudi, another human B lymphoma cell line, human myeloid leukemia cells, HL-60 and OCI-AML3, non-hematologic human cancer cells, HCT-116 (colon cancer) and OVCAR-3 (ovarian cancer), and normal human B cells isolated from healthy donors (FIG. 10A). PVX particles were incubated with cells at 4° C. to assess cell binding while preventing cellular uptake. Post incubation, unbound particles were washed off and flow cytometry was used to detect Cy5 signal and determine the percentage of Cy5 positive cells. Under identical experimental conditions, PVX showed significantly stronger binding with the B cell lymphoma cell lines, Raji and Daudi as compared to the other cell types tested. Specifically, PVX displayed a ˜6 fold higher binding to Raji cells than HL60 cells, OCI-AML3 cells, and non-malignant B cells (CD19⁺ normal B cells), ˜4 fold higher binding than HCT-116 cells and ˜30 fold higher binding than OVCAR-3 cells (FIG. 10A). These results support an intrinsic cellular tropism of PVX towards the malignant B cells.

The potential of utilizing PVX as a targeted drug delivery approach for B cell lymphoma was investigated using MMAE as the therapeutic payload. MMAE is a highly potent antimitotic agent with nearly 100× more potency than anthracyclines such as doxorubicin. MMAE exerts its cytotoxic effects by binding to tubulin, causing G2/M cell cycle arrest, subsequently leading to apoptosis. MMAE also leads to acute systemic toxicity which requires it to be administered as a safer pro-drug. The pro-drug Val-Cit linked MMAE (vcMMAE) is one of the current drugs being tested as a chemotherapeutic payload on ADCs in B cell lymphoma treatment. The vcMMAE formulation is stable under physiologic conditions, but undergoes rapid proteolytic cleavage in the catabolic microenvironment of lysosomes in cancer cells to release the free drug MMAE. Previous data demonstrating endosomal trafficking of PVX supports the choice of this combination of drug payload and nanocarrier. (see Steinmetz, et al., Nano Lett 10(1): 305-312 (2010); and Shukla, et al., Cell Mol Bioeng 8(3): 433-444 (2015)). vcMMAE was conjugated to PVX's cysteine side chain making use of the maleimide handle of the vcMMAE pro-drug (FIG. 10B). A 7500 molar excess of vcMMAE was reacted with PVX overnight at 4° C. (at final concentration of 1 mg mL⁻¹). Post-synthesis, excess unconjugated drugs were separated via ultracentrifugation. Re-suspended PVX-vcMMAE was then purified further by overnight dialysis. PVX stability post drug conjugation was confirmed using transmission electron microscopy (TEM); TEM imaging shows the characteristic protein-based filaments measuring 515×13 nm (FIG. 10C). SDS-PAGE confirmed covalent loading with vcMMAE as evident by a higher molecular weight band, i.e. >25 kDa (which is the native PVX coat protein, CP) (FIG. 10D). Band analysis using the ImageJ software indicated ˜30% of PVX CP conjugated with vcMMAE; or in other words PVX particles were loaded with ˜400 vcMMAE molecules/PVX. We attempted to further increase drug loading, however increasing the vcMMAE/PVX molar ratios during synthesis led to particle aggregations and instability.

Next, the in vitro cytotoxicity of PVX-vcMMAE drug conjugate was verified. When incubated with increasing equivalent concentrations of free pro-drug vcMMAE and PVX-vcMMAE, B lymphoma cells displayed increased cytotoxicity with PVX-vcMMAE as compared to the free drug (FIG. 10E and FIG. 10F). The dose proportional toxicity of PVX-vcMMAE is further supported by the significantly lower IC₅₀ values (50 nM and 23 nM for Raji and Daudi cells, respectively) over the IC₅₀ values of vcMMAE (160 nM and 80 nM in Raji and Daudi cells, respectively) (FIG. 10E and FIG. 10F). As discussed, the pro-drug vcMMAE needs to undergo proteolytic cleavage to release the active form MMAE. As the free pro-drug uptake is limited and inefficient, PVX delivery enables an increase in potency of vcMMAE. It is important to note, that soluble vcMMAE and PVX-vcMMAE both displayed no cytotoxicity towards the non-dividing normal donor B cells. Auristatin drugs only kill rapidly dividing cells by inducing mitotic arrest. (FIG. 10E and FIG. 10F).

Finally, the therapeutic potential of PVX-vcMMAE was evaluated in vivo using a Raji B cell lymphoma model in NSG mice. Male NSG mice were injected intravenously with luciferase expressing Raji cells and monitored for lymphoma engraftment and progression using bioluminescence imaging. Tumor engrafted mice were randomly assigned to the treatment groups (n=5).

On day 3 post-tumor challenges, mice were treated 6 times at four-day intervals with MMAE (0.125 mg/kg), PVX-vcMMAE (0.4 mg/kg), PVX (1 mg—matched to carrier dose of PVX-vcMMAE) or PBS (FIG. 11A). The MMAE dose was selected based on the fact that the known maximum tolerated dose (MTD) of free MMAE is 0.25-0.5 mg kg⁻¹ after a single administration. A dose of 0.125 mg/kg was chosen to allow for multiple treatments. Mice injected intravenously with Raji cells develop hind leg paralysis (due to the infiltration of neoplastic cells into the spinal canal) and/or lose weight prior to death. Therefore, hind leg paralysis or >15% loss in body weight were considered experimental endpoints at which mice were euthanized.

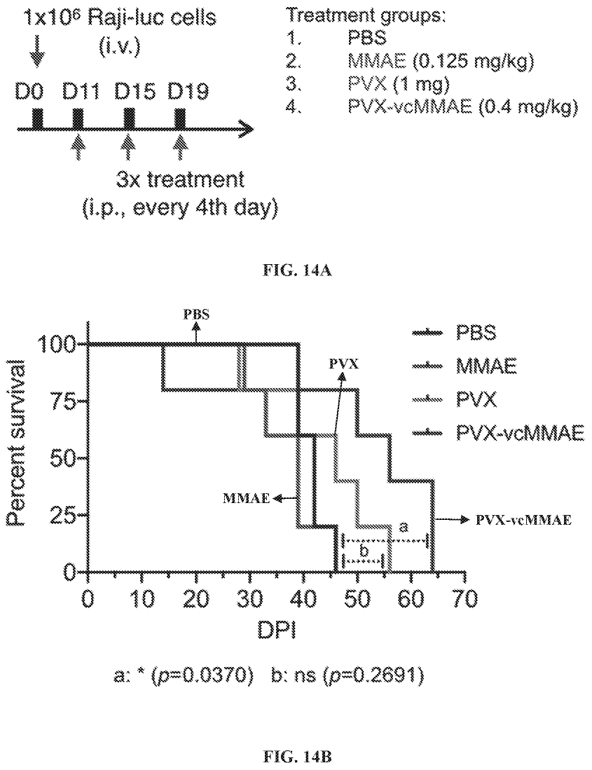

As observed in the biodistribution studies, bioluminescence imaging indicated early signs of lymphoma in kidneys and subsequent dissemination to other regions, such as spleen, lungs and the CNS (FIG. 11B). As evident by bioluminescence intensities, lymphoma progressed rapidly between days 12-24 in the PBS and PVX treatment groups and was widespread by day 38 (FIG. 11C). The median survival for PBS treated mice was 45 days, which is consistent with previous reports. (see Chao, et al., Blood 118(18): 4890-4901 (2011); and Donnou, et al., Adv Hematol 2012: 701704 (2012)) PVX treatment did not improve the survival significantly (median survival of 54 days vs. 45 for PVX treatment; p=0.0527) (FIG. 11D). As evident from bioluminescence imaging and survival data, soluble MMAE treatment partially limited lymphoma progression and improved median survival to 61 days (p=0.009 vs. PBS treatment) (FIG. 11C and FIG. 11D); this limited effect can be explained by the insufficient dosing with soluble MMAE, limited by the inherent toxicities of this drug. In contrast, the PVX-vcMMAE group significantly impaired the progression of lymphoma as evidenced by bioluminescent imaging as well as an increase in overall survival (FIG. 11C and FIG. 11D); the median survival was 74 days (p=0.0134 vs. PBS; p=0.0134 vs. MMAE; p=0.0134 vs. PVX). The efficacy of treatment was dependent number of doses and treatment schedules. In a separate study involving fewer doses and delayed treatment (three doses starting at day 11 post-tumor challenge), the median survival of PVX-vcMMAE treatment group was 56 days vs. 42 days for PBS treated mice (p=0.037) (FIG. 14A and FIG. 14B). In comparison, MMAE and PVX treatment showed no improvement in overall survival to PBS treatment (median survival of 39 days for MMAE and 46 days for PVX with p=0.3513 and p=0.2691 vs. PBS treatment, respectively). Thus, PVX-vcMMAE clearly outperformed MMAE in its soluble form.

Histological analysis performed on mouse kidney sections further corroborated these observations (FIG. 10E and FIG. 10F). Kidneys were harvested from PBS, PVX and MMAE treated mice that were all sacrificed when they became moribund, whereas kidneys from the PVX-vcMMAE treated mice were isolated at the end of study period. Histological sections from the PBS and PVX treatment groups showed the presence of large areas infiltrated by lymphoma cells (dark stained regions) as compared to the MMAE treated group that showed reduced lymphoma cell infiltration. On the other hand, kidney sections from the PVX-vcMMAE treatment group show significantly reduced lymphomas at the end of study period.

In some embodiments as compared to synthetic nanoparticles, PVX nanofilament offers a flexible nanoparticle morphology and higher production yield, as synthetic nanoparticles is difficult to synthesize using purely chemical approaches. Additionally, PVX carries a higher payload compared to ADCs and home to target sites with shorter circulation times, prevents premature release and off-target toxicities.

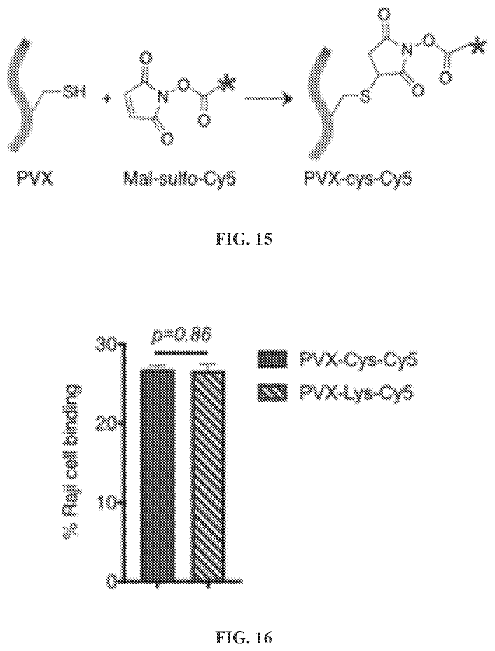

FIG. 15 illustrates PVX nanofilaments conjugated to Cy5 dyes via the Cys residues on the coat protein (CP) using maleimide chemistry.

FIG. 16 illustrates cellular tropism of PVX towards lymphoma cells which was further validated by in vitro cell binding studies using PVX-lys-Cy5 and PVX-cys-Cy5 particles in a panel of cells.

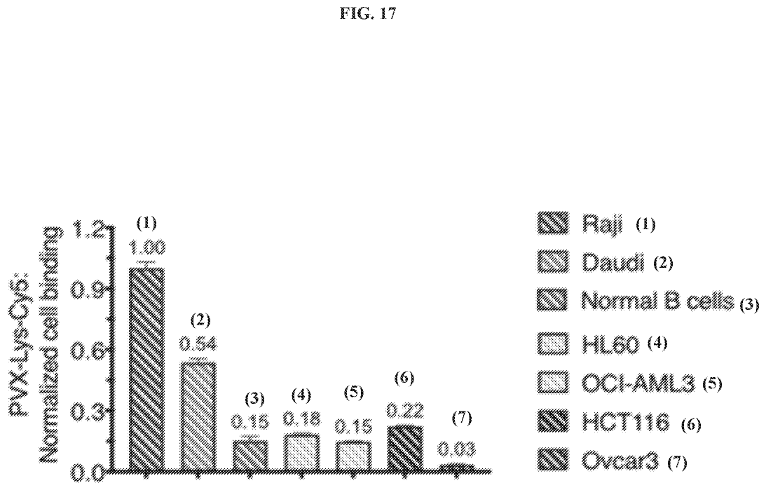

FIG. 17 illustrates in vitro cell binding studies of conjugated PVX in various cell lines. Mean values are provided above bars.

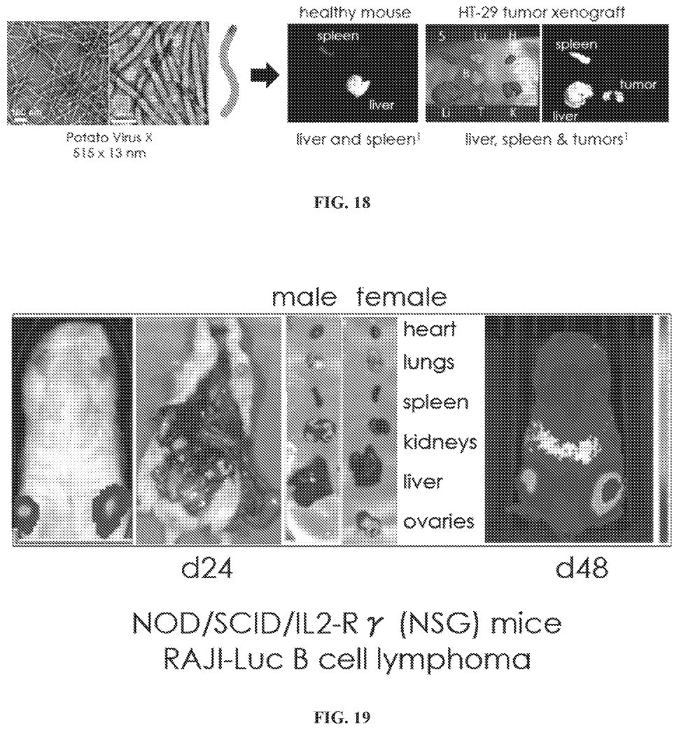

FIG. 18 illustrates PVX nanofilaments biodistribution. PVX nanofilaments were confined to liver and spleens in healthy mice. PVX accumulates in the tumor microenvironment (TME) or homes to the tumor by the enhanced permeability and retention (EPR) effect.

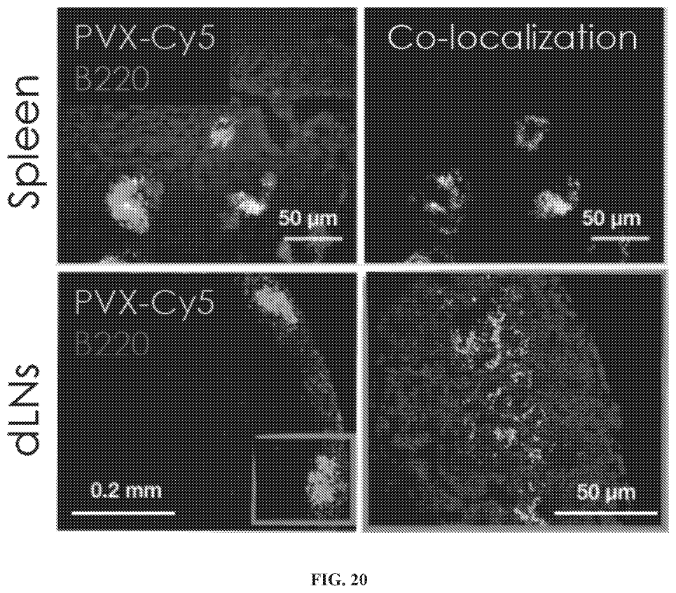

FIG. 19 illustrates systemically disseminated spread of B cell lymphoma following i.v. injection.

FIG. 20 illustrates co-localization of PVX to B-cell rich regions in spleens and draining lymph nodes (dLNs).

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. Cormack, B. P.; Valdivia, R. H.; Falkow, S. Gene 1996, 173, (1 Spec No), 33-8.
2. Banerjee, A. K. Microbiol Rev 1980, 44, (2), 175-205.
3. Sweet, T. J.; Licatalosi, D. D. Methods Mol Biol 2014, 1125, 3-12.
4. Gale, M., Jr.; Tan, S. L.; Katze, M. G. Microbiol Mol Biol Rev 2000, 64, (2), 239-80.
5. Konarska, M. M.; Padgett, R. A.; Sharp, P. A. Cell 1984, 38, (3), 731-6.
6. Munroe, D.; Jacobson, A. Mol Cell Biol 1990, 10, (7), 3441-55.
7. Lam, P.; Gulati, N. M.; Stewart, P. L.; Keri, R. A.; Steinmetz, N. F. Sci Rep 2016, 6, 23803.
8. Arkhipenko, M. V.; Petrova, E. K.; Nikitin, N. A.; Protopopova, A. D.; Dubrovin, E. V.; Yaminskii, I. V.; Rodionova, N. P.; Karpova, O. V.; Atabekov, J. G. Acta Naturae 2011, 3, (3), 40-6.
9. Storni, T.; Ruedl, C.; Schwarz, K.; Schwendener, R. A.; Renner, W. A.; Bachmann, M. F. J Immunol 2004, 172, (3), 1777-85.
10. Azizgolshani, O.; Garmann, R. F.; Cadena-Nava, R.; Knobler, C. M.; Gelbart, W. M. Virology 2013, 441, (1), 12-7.
11. Wiethoff, C. M.; Middaugh, C. R. J Pharm Sci 2003, 92, (2), 203-17.
12. Davis, M. E. Curr Opin Biotechnol 2002, 13, (2), 128-31.
13. Al-Dosari, M. S.; Gao, X. AAPS J 2009, 11, (4), 671-81.
14. Akita, H.; Harashima, H. Expert Opin Drug Deliv 2008, 5, (8), 847-59.
15. Oliveira, S.; van Rooy, I.; Kranenburg, O.; Storm, G.; Schiffelers, R. M. Int J Pharm 2007, 331, (2), 211-4.
16. Hatakeyama, H.; Ito, E.; Akita, H.; Oishi, M.; Nagasaki, Y.; Futaki, S.; Harashima, H. J Control Release 2009, 139, (2), 127-32.
17. Nishimura, Y.; Takeda, K.; Ezawa, R.; Ishii, J.; Ogino, C.; Kondo, A. J Nanobiotechnology 2014, 12, 11.
18. Wadia, J. S.; Stan, R. V.; Dowdy, S. F. Nat Med 2004, 10, (3), 310-5.
19. Akinc, A.; Thomas, M.; Klibanov, A. M.; Langer, R. J Gene Med 2005, 7, (5), 657-63.
20. Sonawane, N. D.; Szoka, F. C., Jr.; Verkman, A. S. J Biol Chem 2003, 278, (45), 44826-31.
21. Curiel, D. T.; Agarwal, S.; Wagner, E.; Cotten, M. Proc Natl Acad Sci USA 1991, 88, (19), 8850-4.
22. Wu, Z.; Chen, K.; Yildiz, I.; Dirksen, A.; Fischer, R.; Dawson, P. E.; Steinmetz, N. F. Nanoscale 2012, 4, (11), 3567-76.
23. Lee, K. L.; Uhde-Holzem, K.; Fischer, R.; Commandeur, U.; Steinmetz, N. F. Methods Mol Biol 2014, 1108, 3-21.
24. Merritt, S. R. S., N. F.; von Recum, H. A. Experimental Biology and Medicine 2017, in press.
25. Chen, X.; Shank, S.; Davis, P. B.; Ziady, A. G. Molecular therapy: the journal of the American Society of Gene Therapy 2011, 19, (1), 93-102.
26. Ugrinova, I.; Petrova, M.; Chalabi-Dchar, M.; Bouvet, P. Adv Protein Chem Struct Biol 2018, 111, 133-164.
27. Huang, X. P.; Wang, X.; Xie, X. L.; Zhang, G. P.; Lv, F. J.; Weng, W. T.; Qiu, F.; Li, Z. F.; Lin, J. S.; Diao, Y. Oncotarget 2018, 9, (2), 2220-2235.
28. Yang, S.; Ren, Z.; Chen, M.; Wang, Y.; You, B.; Chen, W.; Qu, C.; Liu, Y.; Zhang, X. Mol Pharm 2018, 15, (1), 314-325.
29. Liu, M. L.; Zou, H. Y.; Li, C. M.; Li, R. S.; Huang, C. Z. Chem Commun (Camb) 2017, 53, (97), 13047-13050.
30. Li, F.; Lu, J.; Liu, J.; Liang, C.; Wang, M.; Wang, L.; Li, D.; Yao, H.; Zhang, Q.; Wen, J.; Zhang, Z. K.; Li, J.; Lv, Q.; He, X.; Guo, B.; Guan, D.; Yu, Y.; Dang, L.; Wu, X.; Li, Y.; Chen, G.; Jiang, F.; Sun, S.; Zhang, B. T.; Lu, A.; Zhang, G. Nat Commun 2017, 8, (1), 1390.
31. Dhez, A. C.; Benedetti, E.; Antonosante, A.; Panella, G.; Ranieri, B.; Florio, T. M.; Cristiano, L.; Angelucci, F.; Giansanti, F.; Di Leandro, L.; d'Angelo, M.; Melone, M.; De Cola, A.; Federici, L.; Galzio, R.; Cascone, I.; Raineri, F.; Cimini, A.; Courty, J.; Giordano, A.; Ippoliti, R. J Cell Physiol 2018, 233, (5), 4091-4105.
32. Jia, W.; Yao, Z.; Zhao, J.; Guan, Q.; Gao, L. Life Sci 2017, 186, 1-10.
33. Lam, P. Y.; Hillyar, C. R.; Able, S.; Vallis, K. A. J Labelled Comp Radiopharm 2016, 59, (12), 492-499.
34. Stripecke, R.; Cardoso, A. A.; Pepper, K. A.; Skelton, D. C.; Yu, X. J.; Mascarenhas, L.; Weinberg, K. I.; Nadler, L. M.; Kohn, D. B. Blood 2000, 96, (4), 1317-26.
35. Esnault, S.; Malter, J. S. J Immunol 1999, 163, (10), 5228-34.
36. Zhou, Y.; Tang, Z.; Shi, C.; Shi, S.; Qian, Z.; Zhou, S. J Mater Sci Mater Med 2012, 23, (11), 2697-708.
37. Zhou, J.; Yockman, J. W.; Kim, S. W.; Kern, S. E. Pharm Res 2007, 24, (6), 1079-87.
38. Xing, H. B.; Pan, H. M.; Fang, Y.; Zhou, X. Y.; Pan, Q.; Li, D. Oncol Lett 2014, 7, (2), 487-492.
39. Lungwitz, U.; Breunig, M.; Blunk, T.; Gopferich, A. Eur J Pharm Biopharm 2005, 60, (2), 247-66.
40. Chen, J.; Tian, H.; Kano, A.; Maruyama, A.; Chen, X.; Park, T. G. J Control Release 2011, 152 Suppl 1, e134-6.
41. King, D. P.; Montague, N.; Ebert, K.; Reid, S. M.; Dukes, J. P.; Schadlich, L.; Belsham, G. J.; Lomonossoff, G. P. J Virol Methods 2007, 146, (1-2), 218-25.
42. Shukla, S.; Dickmeis, C.; Nagarajan, A. S.; Fischer, R.; Commandeur, U.; Steinmetz, N. F. Biomaterials Science 2014, 2, 784-797.
43. Cruz, S. S.; Chapman, S.; Roberts, A. G.; Roberts, I. M.; Prior, D. A.; Oparka, K. J. Proc Natl Acad Sci USA 1996, 93, (13), 6286-90.
44. Uhde, K.; Fischer, R.; Commandeur, U. Arch Virol 2005, 150, (2), 327-40.
45. Uhde-Holzem, K.; Fischer, R.; Commandeur, U. Arch Virol 2007, 152, (4), 805-11.
46. Uhde-Holzem, K.; Schlosser, V.; Viazov, S.; Fischer, R.; Commandeur, U. J Virol Methods 2010, 166, (1-2), 12-20.
47. Kwoh, D. Y.; Coffin, C. C.; Lollo, C. P.; Jovenal, J.; Banaszczyk, M. G.; Mullen, P.; Phillips, A.; Amini, A.; Fabrycki, J.; Bartholomew, R. M.; Brostoff, S. W.; Carlo, D. J. Biochim Biophys Acta 1999, 1444, (2), 171-90.
48. Pitek, A. S.; Wen, A. M.; Shukla, S.; Steinmetz, N. F. Small 2016, 12, (13), 1758-69.

1. A chimeric Potato virus X (PVX) comprising a nucleic acid encapsulated by a plurality of coat proteins, wherein the nucleic acid comprises one or more of:
a) a non-coding RNA;
b) an mRNA;
c) an siRNA; or
d) an miRNA;
wherein the chimeric PVX homes to the tumor microenvironment (TME) of a B cell malignancy.

2. The chimeric PVX of claim 1, wherein the nucleic acid comprises:
a non-coding RNA, an siRNA, or an miRNA;
an siRNA or an miRNA;
a non-coding RNA;
an mRNA;
an siRNA; or
an miRNA.

3. The chimeric PVX of claim 1, wherein the non-coding RNA is from about 30 nucleotides to about 500 nucleotides in length, optionally from about 30 nucleotides to about 300 nucleotides in length, or from about 50 nucleotides to about 100 nucleotides in length; or wherein the siRNA is from about 8 nucleotides to about 50 nucleotides in length, optionally from about 10 nucleotides to about 30 nucleotides in length, or wherein the miRNA is from about 8 nucleotides to about 50 nucleotides in length, optionally from about 10 nucleotides to about 30 nucleotides in length.

4. The chimeric PVX of claim 1, wherein the mRNA comprises an mRNA coding for a cytokine, an immunomodulatory molecule, or a reporter protein.

5. The chimeric PVX of claim 1, wherein the siRNA downregulates an mRNA that encodes for a protein upregulated in the B cell malignancy.

6. The chimeric PVX of claim 5, wherein the siRNA initiates cell death in a tumor cell of the B cell malignancy.

7. (canceled)

8. The chimeric PVX of claim 1, wherein the miRNA comprises an miRNA that modulates expression of a target protein in a tumor cell of the B cell malignancy.

9. The chimeric PVX of claim 8, wherein the miRNA initiates cell death in the tumor cell.

10. (canceled)

11. (canceled)

12. (canceled)

13. The chimeric PVX of claim 1, wherein the chimeric PVX comprises a therapeutic agent.

14. The chimeric PVX of claim 13, wherein the therapeutic agent is covalently bound to a solvent accessible Lys or Cys residue of a coat protein of the chimeric PVX, optionally through a linker.

15. The chimeric PVX of claim 1, wherein each coat protein of the chimeric PVX comprises at least one therapeutic agent.

16. The chimeric PVX of claim 1, wherein the coat protein is a full-length coat protein.

17. The chimeric PVX of claim 1, wherein the coat protein is a truncated coat protein comprising an N-terminal deletion, optionally comprising a deletion of the first 2, 3, 4, 5, 6, 7, 8, 9, 10, 11, 12, 13, 14, 15, 20, 21, 22, 23, 24, 25, or more residues from the N-terminus.

18. (canceled)

19. The chimeric PVX of claim 1, wherein the therapeutic agent is an anti-cancer drug.

20. The chimeric PVX of claim 19, wherein the anti-cancer drug is a small molecule drug, optionally is MMAE or vmMMAE, and the anti-cancer drug treats the B cell malignancy.

21. (canceled)

22. The chimeric PVX of claim 1, wherein the therapeutic agent is an anti-cancer peptide or protein.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A host cell comprising the chimeric PVX of claim 1.

37. (canceled)

38. (canceled)

39. (canceled)

40. A method of producing a chimeric PVX comprising culturing the host cell of claim 1.

41. A method of treating or inhibiting the progression of a hematologic malignancy in a subject in need thereof, comprising:
administering to the subject the chimeric PVX of claim 1.

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. A kit comprising one or more of: the chimeric PVX of claim 1.

57. (canceled)
